(19) United States
(12) Patent Application Publication
(10) Pub. No.: US 2026/0115283 A1
(43) Pub. Date: Apr. 30, 2026

(54) METHOD FOR TREATING CANCER BY USING COMBINATION OF ASPARAGINE INHIBITOR AND IMMUNE CHECKPOINT INHIBITOR AND PHARMACEUTICAL COMPOSITION COMPRISING COMBINATION OF ASPARAGINE INHIBITOR AND IMMUNE CHECKPOINT INHIBITOR

(71) Applicant: CHANG GUNG MEMORIAL HOSPITAL, LINKOU, TAOYUAN CITY (TW)

(72) Inventors: Huang-Yu Yang, Taoyuan City (TW); Ping-Chih Ho, Taoyuan City (TW); Drew Mark Pardoll, Taoyuan City (TW); Cheng-Ta Yang, Taoyuan City (TW); Chun-Te Wu, Taoyuan City (TW); See-Tong Pang, Taoyuan City (TW); Cheng-Lung Hsu, Taoyuan City (TW); Hsuan-Chia Chang, Taoyuan City (TW); Chung-Ying Tsai, Taoyuan City (TW)

(21) Appl. No.: 19/219,665

(22) Filed: May 27, 2025

Related U.S. Application Data
(60) Provisional application No. 63/652,187, filed on May 28, 2024.

(51) Int. Cl. A61K 39/395; A61K 38/50; A61K 39/00; A61P 35/00; A61P 35/04; C07K 16/28

(52) U.S. Cl. CPC A61K 39/3955; A61K 38/50; A61P 35/00; A61P 35/04; C07K 16/2818; C12Y 305/01001; A61K 2039/505; C07K 2317/76

(57) ABSTRACT

The present disclosure provides a method for treating cancer by using a combination of an asparagine inhibitor and an immune checkpoint inhibitor and a pharmaceutical composition including the combination of the asparagine inhibitor and the immune checkpoint inhibitor. The present disclosure achieves the effects of treating cancer through various efficacy experiments.

### CROSS-REFERENCE TO RELATED APPLICATION

This application claims priority of Provisional application No. 63/652,187, filed on May 28, 2024, the content of which is incorporated herein in its entirety by reference.

### BACKGROUND OF THE INVENTION

### 1. Field of the Invention

The present invention relates to a method for treating cancer by using a combination of an asparagine inhibitor and an immune checkpoint inhibitor and a pharmaceutical composition comprising the combination of the asparagine inhibitor and the immune checkpoint inhibitor.

### 2. The Prior Art

Cancer is one of the main causes of human death today. Although the mechanism of cancer formation is still not fully understood, carcinogenesis or tumorigenesis can be attributed to the accumulation of exogenous or endogenous factors leading to genetic abnormalities in the cells, the signal transduction pathways in these cells would be wrong, causing cell division to be out of control, and then these cells would gradually form cancer cells. Cancer cells can avoid apoptosis and have the ability of migration and invasion. Therefore, cancer cells would continue to proliferate and metastasize to other parts of the body via the lymphatic system or vascular system.

Although there are many drugs currently used in clinical treatment of cancer, the therapeutic effect is still not ideal. The main reasons include: individual differences in patients themselves, serious side effects of anti-cancer drugs, and drug resistance of cancer cells. Therefore, researchers in the art are committed to the development of effective drugs that do not produce undesirable side effects to treat cancer. On the other hand, in addition to generally suppressing the proliferation of cancer cells, suppressing the metastasis of cancer cells is also a very important and difficult problem to be solved.

Immune checkpoint inhibitors (ICIs) have emerged as a promising therapeutic strategy to re-evoke anti-tumor immunity by targeting the immunocheckpoint pathways. However, the efficacy of ICIs can be limited by the suppressive nature of the tumor microenvironment that prevents T cell tumor infiltration and T cell dysfunction. Among chemotherapy drugs. L-asparaginase is an enzymatic drug that has been approved by treating for acute lymphoblastic leukemia (ALL) and myeloid leukemia therapy. However, the therapeutic potential of asparaginase in solid tumors remains elusive.

In order to solve the above-mentioned problems, those skilled in the art urgently need to develop a novel, safe and effective pharmaceutical composition for treating cancer for the benefit of a large group of people in need thereof.

### SUMMARY OF THE INVENTION

In order to solve the foregoing problems, an objective of the present invention is to provide a method for treating cancer, comprising administering to a subject in need thereof a pharmaceutical composition comprising an effective amount of a combination of an asparagine inhibitor and an immune checkpoint inhibitor (ICI).

Another objective of the present invention is to provide a pharmaceutical composition for treating cancer, comprising a combination of an asparagine inhibitor and an immune checkpoint inhibitor (ICI).

According to an embodiment of the present invention, the cancer is bladder carcinoma, breast cancer, glioblastoma, nasopharyngeal carcinoma, lung cancer, colorectal cancer, liver cancer, urothelial cancer, pancreatic cancer, renal cell carcinoma, prostate cancer, oral cancer, or melanoma.

According to an embodiment of the present invention, the nasopharyngeal carcinoma is recurrent-metastatic nasopharyngeal carcinoma (RM-NPC).

According to an embodiment of the present invention, the asparagine inhibitor is L-asparaginase.

According to an embodiment of the present invention, the immune checkpoint inhibitor is an anti-PD-I monoclonal antibody.

According to an embodiment of the present invention, the anti-PD-1 monoclonal antibody is pembrolizumab.

According to an embodiment of the present invention, the combination of the asparagine inhibitor and the immune checkpoint inhibitor enhances efficacy of cancer immunotherapy by activating killer T cells.

According to an embodiment of the present invention, the killer T cells are CD8⁺ T cells.

According to an embodiment of the present invention, the asparagine inhibitor has an asparagine deprivation effect.

According to an embodiment of the present invention, the asparagine deprivation enhances glutamine metabolism to enhance CD8⁺ T cell activation.

According to an embodiment of the present invention, the asparagine deprivation tailors differentiation and metabolic adaptation in a reactive oxygen species (ROS)-dependent manner.

According to an embodiment of the present invention, the asparagine deprivation enhances CD8⁺ T cell effector functions.

According to an embodiment of the present invention, the pharmaceutical composition is in a dosage form for oral administration.

According to an embodiment of the present invention, the pharmaceutical composition is in a dosage form for parenteral administration.

In summary, the results of the present invention are illustrated by the following examples. The present invention discovered that asparagine inhibitory agent can activate cytotoxic T cells, promote the killing effect of cytotoxic T cells on tumors. Clinical and animal experiments have confirmed that asparagine inhibitory agent combined with immune checkpoint inhibitors can enhance the efficacy of cancer immunotherapy by activating killer T cells. The invention proposes a combination therapy using asparagine inhibitory agent and immune checkpoint inhibitors, providing a new strategy for cancer immunotherapy development.

The combination of asparaginase and anti-PD-1 monoclonal antibody (mAb) has shown synergistic anti-tumor effects in murine tumor models. In compassionate therapy for recurrent-metastatic nasopharyngeal carcinoma (RM-NPC) patients, the present invention observed that the combined treatment with L-asparaginase and PD-1 blockade mAb induced a robust anti-tumor response dependent on CD8⁺ T cell-mediated immune activity. Further investigation revealed that prolonged asparagine deprivation dysregulates mitochondrial complex I subunit expression, promoting reactive oxygen species (ROS) production and enhancing glutamine metabolism. This ultimately guides nuclear factor of activated T-cells (NFAT) nuclear localization and activity via ROS signaling.

The present invention found that asparaginase combined with the anti-PD-1 antibody elicited robust anti-tumor responses in NPC patients accompanied with inflammatory signs. By combining multi-dimensional flow analysis in both NPC patients and tumor-bearing murine models, we unveiled that asparaginase treatment modulated CD8⁺ T cell differentiation state and stimulated T cell-dependent anti-tumor immunity in combination with the anti-PD-1 antibody.

The present invention can provide a new combination strategy for cancer immunotherapy in clinical medicine, using the combination therapy of an asparagine inhibitor and an immune checkpoint inhibitor to improve the success rate of cancer immunotherapy.

The L-asparaginase drug Leunase® is a clinically used drug for the treatment of leukemia. Through the combined treatment strategy of the present invention, asparaginase can be used in the treatment of cancer patients with solid tumors, such as bladder carcinoma, breast cancer, glioblastoma, nasopharyngeal carcinoma, lung cancer, colorectal cancer, liver cancer, urothelial carcinoma, pancreatic cancer, renal cell carcinoma, prostate cancer, etc.

The embodiments of the present invention would be further described below. The following examples are used to illustrate the present invention and are not intended to limit the scope of the present invention. Anyone skilled in the art can make some changes and modifications without departing from the spirit and scope of the present invention. Therefore, the scope of the present invention shall be defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included here to further demonstrate some aspects of the present invention, which can be better understood by reference to one or more of these drawings, in combination with the detailed description of the embodiments presented herein.

FIGS. 1A-1D show that asparaginase synergize with anti-PD-I to elevate T cell anti-tumor responses. (1A) B16-F10-Luc-bearing C57BL/6 and Tcrb KO mice were administered with vehicle or L-asparaginase (5 U/g) three days before tumor inoculation and then 3 times per week by intraperitoneal injection. The average luminescent intensity of photons emitted from each tumor in the images was quantified. Data are presented as the mean±SEM (n=4 for each group). (1B) B16-OVA-bearing C57BL/6 mice were administered with vehicle or L-asparaginase (5 U/g) or PBS on day 7 after tumor inoculation and received 1.5×10\hat{ }6 activated OTI T cells on day 10. The average luminescent intensity of photons emitted from each tumor in the images was measured. Error bars represent the standard error of the mean (SEM). Data is representative of one independent experiment with n=3 mice per group. (1C) MTCQ1-bearing C57BL/6 were treated with either a vehicle, L-ASP, anti-PD-1, or a combination of L-ASP and anti-PD-1 starting on day 7 after tumor inoculation. The average tumor volume is emitted from each tumor group. (1D) B16-OVA-bearing C57BL/6 mice were treated with either a vehicle, L-ASP, anti-PD-1, or a combination of L-ASP and anti-PD-1 starting on day 7 after tumor inoculation. The average luminescent intensity of photons and tumor volume are emitted from each tumor group.

FIGS. 2A-2D show that asparaginase boost PD-1 blockade-induced CD8⁺ T cell activation. (2A) tSNE projection of Tcrb⁺CD45⁺CD8⁺ TIL clusters identified by FlowSOM clustering and percentage of TIL from different groups in each FlowSOM cluster (n=3). (2B) FlowSOM clusters, with a heat map of effector molecule MFIs overlaid onto the t-SNE analysis. (2C) tSNE projection of CD8⁺ T cells for all groups with different treatments. (2D) tSNE projections of indicated protein expression in FIG. 2A. All data are mean±SD and were analyzed by two-tailed unpaired Student's t-test. NS, not significant.

FIGS. 3A-3D show that asparagine deprivation enhances CD8⁺ T cell effector functions. Naïve CD8⁺ T cells were isolated from WT B6 mice and cultured under CD8 differentiation conditions using a medium without asparagine (Asn-free) or with asparaginase (L-ASP). (3A) After three days of culture, the cells were re-stimulated with PMA and ionomycin for four hours in the presence of Golgi stop, permeabilized, and stained for IFN-γ and granzyme B. (3B) GSEA tracing for T cell activation in Asn-free and control cultured CD8⁺ T cell. ES, enrichment score; NES, normalized enrichment score; FDR, false discovery ratio. (3C) human CD8⁺ T cells were isolated from human PBMCs using the Naive CD8⁺ T Cell Isolation Kit, human. CD8⁺ T cells cultured under control and asparagine-deprived conditions. After 3 days cultured, cells were then re-stimulated with PMA and ionomycin for 4 hours with Golgi stop, permeabilized and stained for IFN-γ and granzyme B. (3D) tSNE projections of expression of the indicated proteins. All data are mean±SD and were analyzed by two-way ANOVA with Sidak multiple comparisons tests with adjusted P value and data are cumulative results from at least three independent experiments.

FIGS. 4A-4E show that asparaginase boost PD-1 blockade-induced anti-tumor immunity. (4A) Schematic diagram illustrating the design of a clinical trial for Nasopharyngeal carcinoma (NPC) patients. (4B) t-SNE analysis of CD45⁺CD8⁺ T cells from PBMC populations (n=8) identifies distinct. Percentage of CD45⁺CD8⁺ T cells from different days in each FlowSOM cluster. (4C) FlowSOM clusters, with a heat map of effector molecule MFIs overlaid onto the t-SNE analysis. (4D) PET scans of 18F-FDG in patients with NPC and the maximum standardized uptake value (SUVmax) before and after receiving asparaginase and pembrolizumab combination therapy and (4E) plasma EBV relative DNA copy number distribution in patients with metastatic NPC conduct combination therapy (6 patients) and PD-1 therapy (2 patients). All data are mean±SD and were analyzed by two-tailed, paired Student's t-test. NS, not significant.

FIGS. 5A-5C show that asparagine deprivation enhances CD8⁺ T cell effector functions. Naïve CD8⁺ T cells were isolated from WT B6 mice and cultured under CD8 differentiation conditions using a medium without asparagine (Asn-free). For various durations, the cells were re-stimulated with PMA and ionomycin for four hours in the presence of Golgi stop, permeabilized, and stained for IFN-γ and granzyme B. (5A) t-SNE analysis of CD8⁺ T cells in differentiation conditions using both control medium and asparagine-deprived medium (5B) with a heat map of effector molecule MFIs overlaid onto the t-SNE analysis. (5C) tSNE projection of CD8⁺ T cells for all groups with various durations and percentages of CD8⁺ T cells from different days in each FlowSOM cluster (n=3). All data are mean±SD and were analyzed by two-way ANOVA with Sidak multiple comparisons tests with adjusted P value and data are cumulative results from at least three independent experiments.

FIGS. 6A-6C show that asparagine deprivation reprograms mitochondrial fitness in T cells. (6A) GSEA tracing for mitochondrial biogenesis in Asn-free and control cultured CD8⁺ T cell. ES, enrichment score: NES, normalized enrichment score; FDR, false discovery ratio. (6B) The level of indicated mRNAs in control T cells and asparagine-free T cell were determined by qPCR. (6C) Naïve CD8⁺ T cells cultured in asparagine-free and control medium for several days and stained with MitoTracker™ Green FM Dye and MitoTracker™, Deep Red FM Dye. Student's t-test or two-way ANOVA with Sidak multiple comparisons tests with adjusted P value (6B, 6C) and data are cumulative results from at least three independent experiments. NS, not significant.

FIGS. 7A-7C show that asparagine deprivation influences metabolic preferences in T cells. (7A) Traces of oxygen consumption rates (OCRs) were recorded. The following compounds were injected into the assay micro-chambers as indicated: oligomycin (Olig.), carbonyl cyanide-4-(trifluoromethoxy)phenylhydrazone (FCCP), and rotenone plus antimycin A (R+A). (7B) Traces of extracellular acidification rates (ECARs) were recorded. Compounds injected included glucose (Glc), oligomycin, and 2-deoxy glucose (2DG). Each data point represents the mean±standard deviation (SD) of four technical replicates. (7C) Lysed cells from a control medium or an asparagine-deprived medium 3-day cultured CD8⁺ T cell. The cells were subsequently analyzed using liquid chromatography-mass spectrometry (LC-MS) to assess the glycolysis (right) and TCA cycle (left). Student's t-test or two-way ANOVA with Sidak multiple comparisons tests with adjusted P value and data are cumulative results from at least three independent experiments. NS, not significant.

FIGS. 8A-8D show that asparagine deprivation affects glutamine metabolism to enhance CD8⁺ T cell activation. (8A) Lysed cells from a control medium or an asparagine-deprived medium 3-day cultured CD8⁺ T cell. The cells were subsequently analyzed using liquid chromatography-mass spectrometry (LC-MS) to assess the glutamine and glutamate. (8B) GSEA tracing for mitochondrial biogenesis in Asn-free and control cultured CD8⁺ T cell. ES, enrichment score; NES, normalized enrichment score; FDR, false discovery ratio. (8C) The level of indicated mRNAs in control T cells and asparagine-free T cell were determined by qPCR. (8D) Naïve CD8⁺ T cells were cultured in CD8 differentiation conditions with either asparagine-free or control medium for three days. For the Asn-free to DF condition (Asparagine and Glutamine-free), after two days of culture in an asparagine-free medium, the cells were transferred to a medium lacking both asparagine and glutamine for one day and then stained for IFN-γ and granzyme B by flow cytometry. All data are mean±SD and were analyzed by two-tailed paired Student's t-test or two-way ANOVA with Sidak multiple comparisons tests with adjusted P value and data are cumulative results from at least three independent experiments.

FIGS. 9A-9E show that asparagine deprivations modulates chromatin accessibility pattern in T cells. (9A) The signal of open chromatin in both Asn-free and control conditions on Day 3 are represented in the genomics locus of IFNG and GZMB. The orange indicated the Asn-free condition whereas the blue indicated the control condition. (9B, 9C) The level of indicated mRNAs in T cells cultured under control and asparagine-deprived conditions in the presence of N-acetylcysteine (NAC) were determined by qPCR. (9D) B16-OVA-bearing Tcrb KO mice were received 1.5×10\hat{ }6 activated OTI T cells on day 7. Data are presented as the mean±SEM (n=5 for each group). (9E) human CD8⁺ T cells were isolated from human PBMCs using the Naive CD8⁺ T Cell Isolation Kit, human. CD8⁺ T cells cultured under control and asparagine-deprived conditions in the presence of N-acetylcysteine (NAC) for 3 days. Student's t-test and data are cumulative results from at least three independent experiments. NS, not significant.

FIGS. 10A-10G show that asparagine deprivation tailors differentiation and metabolic adaptation in a ROS-dependent manner. (10A) The enriched TFs in both Asn-free and control conditions are selected, and the TFs that have the most target region enrichment in either Asn-free or control are represented as the preference. (10B, 10C) Naïve CD8⁺ T cells were cultured under CD8 differentiation conditions in both control medium and asparagine-deprived medium for various durations. Subsequently, the cells were stained for DCFDA and MitoSOX. (10D) Lysed cells from a control medium or an asparagine-deprived medium 3-day cultured CD8⁺ T cell. The cells were subsequently analyzed using liquid chromatography-mass spectrometry (LC-MS) to assess GSSG/GSH ratio. (10E) Naïve CD8⁺ T cells were cultured under CD8 differentiation conditions in both control medium and asparagine-deprived medium for various durations. The protein levels of the indicated molecules were determined through immunoblot analysis. (10F) Naïve CD8⁺ T cells cultured in CD8 differentiation conditions with either asparagine-free or control medium and added with 20 mM NAC for three days, then stained for IFN-γ and granzyme B by flow cytometry. (10G) Imaging of NFAT localization staining (in green) and DAPI (in blue) by confocal microscopy. All data are mean±SD and were analyzed by two-tailed paired (10D) Student's t-test, one-way ANOVA with two-way ANOVA with Tukey's multiple comparisons test (10F, 10G) or Sidak multiple comparisons tests with adjusted P value (10B, 10C) and data are cumulative results from at least three independent experiments.

FIGS. 11A-11D show that the synergistic effect of asparaginase and anti-PD-1 in specific tumor type. (11A) MB49 bladder carcinoma (11B) E0771 breast cancer (11C) LLC1 lung carcinoma (11D) GL261 glioblastoma-bearing C57BL/6 were treated with either a vehicle, L-ASP, anti-PD-1, or a combination of L-ASP and anti-PD-1 starting on day 7 after tumor inoculation. The average tumor volume is emitted from each tumor group.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

As used herein, the term “treating” or “treatment” refers to alleviating, reducing, ameliorating, relieving, or controlling one or more clinical signs of a disease or disorder, and lowering, stopping, or reversing the progression of severity regarding the condition or symptom being treated.

According to the present invention, solid tumors include, but are not limited to, nasopharyngeal carcinoma, lung cancer, colorectal cancer, liver cancer, urothelial cancer, pancreatic cancer, renal cell carcinoma, prostate cancer, oral cancer, and melanoma.

The pharmaceutical composition provided according to the present invention can be in any suitable form without special limitations. It depends on the desired use and can be in a corresponding suitable dosage form. For example, but not limited thereto, the pharmaceutical composition can be administered to subjects in need via oral or parenteral administration.

According to the present invention, the pharmaceutical composition can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

The pharmaceutical composition according to the present invention can comprise a pharmaceutically acceptable carrier which is widely used in pharmaceutical manufacturing technology. For example, the pharmaceutically acceptable carrier can comprise one or more reagents selected from the group consisting of solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. The selection and quantity of these reagents fall within the scope of the professional literacy and routine techniques of those skilled in the art.

According to the present invention, the pharmaceutically acceptable vehicle comprises a solvent selected from the group consisting of water, normal saline, phosphate buffered saline (PBS), sugary solution, aqueous solution containing alcohol, and combinations thereof.

The pharmaceutical composition provided according to the present invention can be administered with different administration frequencies such as once a day, multiple times a day, or once a few days, depending on the individual's needs, age, weight and health status. In the pharmaceutical composition provided according to the present invention, the content ratio in the pharmaceutical composition can be adjusted according to actual application requirements. In addition, the pharmaceutical composition may additionally contain one or more other active ingredients (such as cancer treatment drugs) as needed, or be used in combination with drugs containing the one or more other active ingredients in order to further strengthen the effect of the pharmaceutical composition or increase the flexibility and deployment degree of the formulation, as long as the other active ingredients have no adverse effect on the benefits of the active ingredients of the present invention (i.e., the combination of an asparagine inhibitor and an immune checkpoint inhibitor (ICI)).

The present invention is further illustrated by the following examples. These examples are provided for illustration only and are not intended to limit the scope of the present invention. The scope of the present invention is shown in the appended claims.

The procedures regarding human subjects are as follows. The following example was a single-center, prospective salvage therapy conducted under compassionate-use conditions, employing L-asparaginase for patients with refractory nasopharyngeal carcinoma (NPC) who did not respond to standard treatment. Patients visiting the Oncology Department at Linkou Chang Gung Memorial Hospital between August 2023 and March 2024 were screened. Nine patients with standard treatment-refractory NPC were identified, with one excluded due to an autoimmune disease (see Table 1). Eight patients were ultimately enrolled. Six patients opted for combination salvage therapy, while two chose to continue with anti-PD1 monotherapy. Baseline demographic and clinical characteristics for all eight patients are now included in the revised manuscript. In the combination therapy group, all patients received a single course of 10,000 IU/m² L-asparaginase for three or five days prior to the ongoing anti-PD1 therapy. Apart from this single course of L-asparaginase, cancer treatment and follow-up procedures were consistent with those employed for NPC patients at Chang Gung Memorial Hospital, including PET/CT follow-up, biochemical lab tests, and EBV DNA titer monitoring.

TABLE 1

| | Combination therapy | | | | | | Anti-PD1 alone | |
|---|---|---|---|---|---|---|---|---|
| Case | 1 | 2 | 3 | 4 | 5 | 6 | 7 | 8 |
| Age | 50-54 | 55-60 | 35-39 | 50-54 | 30-34 | 50-54 | 55-60 | 65-69 |
| Gender | Male | Male | Male | Male | Male | Female | Female | Male |
| NPC | T4N1M0 | T4N2M0 | T4N2M1 | T4N1M0 | T1N2M0 | T3N3M1 | T3N2M1 | T4N3M1 |
| stage | stage IVa | stage IVa | Stage IVb | stage IVa | stage III | Stage IVb | Stage IVb | Stage IVb |
| (initial) | | | | | | | | |
| NPC | CCRT | Gem + | Pembo | CCRT | CCRT | Gem + Cis | CCRT | Gem + Cis |
| treatment | Gem + Cis | Carbo | Gem + Cis | Cis + 5FU | Gem + Cis | Taxo + Cis | Gem + Cis | Chemo + |
| (Before | Taxo + Cis | T + Carbo | Taxo | Taxo + Cis | Pembo | Cetuximab | Carbo | Pembo |
| Asparaginase | Pembo | Chemo + | | Xeloda | (clinical | Pembo | Pembo | Taxo + |
| or ICIs | (clinical | Pembo | | Pembo | trail) | Taxo | Chemo + | Carbo |
| therapy) | trail) | 5-FU | | Chemo + | | Chemo + | Pembo | Gem + Cis |
| | | | | Pembo | | Pembo | Taxo | |
| | | | | Gem + | | | | |
| | | | | Carbo | | | | |
| NPC | T0N0M1 | T3N0M1 | T4N2M1 | T0N0M1 | T0N0M1 | T3N3M1 | T4N2M1 | T4N3M1 |
| stage | Stage IVb | Stage IVb | Stage IVb | Stage IVb | Stage IVb | Stage IVb | Stage IVb | Stage IVb |
| (Before | | | | | | | | |
| Asparaginase | | | | | | | | |
| or ICIs | | | | | | | | |
| therapy) | | | | | | | | |
| Metastasis | Liver and | Liver | Liver | Lung, | Lymph | Lung and | Liver and | Liver and |
| (Before | bone | | | bone, | node | bone | lung | bone |
| Asparaginase | | | | Lymph | lung | | | |
| or ICIs | | | | node and | | | | |
| therapy) | | | | liver | | | | |
| Smoking | Yes | Yes | No | No | Yes | No | No | No |
| Betel | Yes | No | No | No | Yes | No | No | No |
| nut | | | | | | | | |
| Underlying | HBV | DM (Diet | Nil | DM, HTN, | Nil | HBV | Nil | BPH |
| disease | | control) | | gout | | | | |
| Follow | 202104 | 202009 | 202209 | 201602 | 2021 | 201911 | 202205 | 202302 |
| up | diagnosis | diagnosis | diagnosis | diagnosis | diagnosis | diagnosis | diagnosis | diagnosis |
| period | 202110 | 202208 | 202203 | 202203 | 2019 | 2019 ICI | 202207 | Liver and |
| after | recurrence | liver meta | recurrence | recurrence | recurrence | 202003 | Liver and | bone meta |
| NPC | 202201 | | 202212 | | 2019 ICI | lung meta | lung meta | |
| diagnosis | liver meta | | liver meta | | | | | |
| | 202205 | | | | | | | |
| | bone meta | | | | | | | |
| Follow | 202308 | 202312 | 202403 | 202403 | 202402 | 202403 | 202206- | 202302 |
| up | combination | combination | combination | combination | combination | combination | 202301 | Chemo + |
| period | therapy | therapy | therapy | therapy | therapy | therapy | Pembo | Pembo |
| after | | | | | | | 202303- | |
| asparaginase | | | | | | | 202312 | |
| or ICIs | | | | | | | Chemo + | |
| | | | | | | | Pembo | |
| therapy | | | | | | | | |
| combination | | | | | | | | |
| or | | | | | | | | |
| monotherapy^{#} | | | | | | | | |
| Image | Decrease | Decrease | Decrease | Decrease | Decreased | Decreased | Increased | Increased |
| evaluation | SUVmax | SUVmax | SUVmax | SUVmax | tumor | tumor | SUVmax | SUVmax |
| after | | | | | length in | length in | | |
| combination | | | | | CT | CT | | |
| therapy | | | | | | | | |
| Mortality | 202312 | | | | | | | |
| | died | | | | | | | |
| Image | Immune | Immune | Immune | Immune | Immune | Immune | Unconfirmed | Unconfirmed |
| evaluation | stable | partial | complete | partial | partial | partial | progression | progression |
| by | disease | response | response | response | response | response | | |
| iRECIST | | | | | | | | |

The procedures regarding in vitro T cell activation studies are as follows. Naïve CD8⁺ T cells were isolated with naïve CDS adverse selection cocktail and LS columns (Miltenyi) from C57BL/6 wild-type mice splenocytes and lymph nodes. One million cells/mL in a 24-well plate were activated with plate-bound anti-CD3 (5 μg/mL) with soluble anti-CD28 (2 μg/mL) and IL-2 (10 ng/mL) in control or asparagine deprivation medium. For restimulation, approximately 1×10⁶ cells per well in a 96-well plate were stimulated in the presence of GolgiStop (BD Bioscience) for four h at 37° C. with PMA (50 ng/ml) and Ionomycin (500 ng/ml). PBMC from healthy voluntary donors were isolated by density gradient centrifugation on Ficoll Paque® according to the manufacturer's instructions (Pharmacia, Uppsala, Sweden). Human CD8⁺ T cells were isolated from human PBMCs using the Naive CD8⁺ T Cell Isolation Kit, human (Miltenyi Biotec). One million cells/mL in a 24-well plate were activated with anti-CD3 (10 ng/ml), soluble anti-CD28 (2 μg/mL) and IL-2 (40 U/ml; Genzyme, Cambridge, MA, USA), plate-bound anti-CD3 (5 μg/mL) with soluble anti-CD28 (2 μg/mL) and IL-2 (10 ng/mL) in control or asparagine deprivation medium.

The procedures regarding tumor growth and survival experiments are as follows. Tumor injections were administered on the right flank. C57BL/6 wild-type mice were injected with 5×10⁵ tumor cells (s.c.) cultured in a DMEM-based medium. L-asparaginase was dissolved in PBS and administered for all vehicle-treated control experiments. Groups were randomized based on tumor size on the day of beginning treatment. Mice were treated with L-asparaginase (5 U/g) or vehicle three times per week. For combination L-asparaginase and immune checkpoint experiments, anti-PD-1 mAb (RMP1-14, Bio X cell) was therapy experiments in the OVA-expressing B16 melanoma model, C57BL/6 wild-type mice received an injection of 5×10⁵ B16-OVA-Luc melanoma cells (s.c.) cultured under OVA selection media containing 400 μg/ml G418 (Life technologies). Mice were treated with L-asparaginase or vehicle thrice weekly in 100 μL for days 7-9 post-tumor inoculation. Ten days after tumor injection, mice received an adoptive transfer of 1.5×10⁶ activated OTI cells (r.o.). Mice were randomized based on tumor size before initiating anti-PD-1 therapy or transferring activated OTI cells in the case of adoptive transfer experiments. Tumor burden was assessed every 2-4 days by measuring the length and width of the tumor. Tumor volume was calculated using the formula V=(L×W×W)/2, where V is tumor volume, W is tumor width, and L is tumor length. For IVIS Imaging, Luciferin, the substrate for luciferase, was administered intraperitoneally at a dose of 100 mg/kg body weight. After injection, mice were anesthetized and positioned on the imaging stage of the IVIS apparatus in the abdominal (subcutaneous model) position. Images were acquired every few minutes from 10 to 30 minutes post-injection using the IVIS® Spectrum 3D system. Photon emission from the tumor and surrounding tissues was quantified using Living Image Software (Aura Imaging Software v 4.0). Mice were sacrificed when the tumor reached 2 cm in any dimension, became ulcerate or necrotic, or caused functional deficits. Unless otherwise specified, average tumor volume is depicted until the first sacrifice.

The procedures regarding tumor-infiltrating lymphocyte (TIL) isolation are as follows. Tumors were harvested from sacrificed mice on day 18 post-tumor inoculation. Isolated tumors were incubated in collagenase type I (Gibco) and DNase (Roche) in RPMI for 30 min at 37° C. Tumor mixtures and spleens were dissociated through a 70-μm filter and washed with PBS. Tumor suspensions were pelleted and resuspended in 40% Percoll solution, underlaid with 80% Percoll in a 15 mL conical flask. After centrifugation at 2000 g for 30 min, the middle layer was removed, washed in PBS, and counted. For in vitro stimulation assays, approximately 1×10⁶ cells per well in a 96-well plate were stimulated in the presence of GolgiStop (BD Bioscience) for four h at 37° C. with PMA (50 ng/ml) and lonomycin (500 ng/ml).

The procedures regarding FACS analysis and antibodies are as follows. The following clones of antibody were purchased from Biolegend (San Diego, CA, USA) or BD Bioscience (San Jose, CA, USA) and used for cell surface staining. For the human panel: LAG-3 (11C3C65), CD279 (EH12.1), CD8 (RPA-T8), ICOS (DX29), CD4 (SK3), Tim3 (7D3), CD3 (UCHT1), CD152 (BN13), CD45RA (HI100), KLRG1 (13F12F2), CD69 (FN50), CD127 (A019D5), CD25 (2A3), TIGIT (A15153G), CD197 (150503), CD45RO (UCHL1). For mouse panel: CD25 (PC61), TCRb (H57-597), TIGIT (1G9), CD366 (5D12), Ly6C (HK1.4), CD44 (1M7), CD223 (C9B7W), CD279 (29F.1A12), CD152 (UC10-4B9), KLRG1 (2F1), CD62L (MEL-14), CD127 (A7R34). For nuclear protein staining. Cells were fixated and permeabilized using the FOXP3 transcription factor staining kit (Thermo Fisher Scientific, Waltham, MA, USA) according to the manufacturer's instructions. For the human panel: IFNg (B27), Il2 (MQ1-17H12), GranB (GB11), TCF7 (S33-966), TOX (TXRX10), T-bet (4B10), TNF-a (MAb11). For mouse panel: TNF-α (MP6-XT22), Rorgt (Q31-378), CD45 (30-F12), TCF-7 (S33-966), EOMES (Dan11mag), IL2 (JES6-5H4), IFNg (XMG1.2) were purchased from Biolegend or BD Bioscience. Flow cytometry was performed on a BD FACSymphony™ A5 Cell Analyzer or BD FACSVerse™ Cell Analyzer and analyzed with FlowJo software (version 10.8.1).

The procedures regarding metabolic assays are as follows. For Seahorse metabolic flux analyses, isolated CD8⁺ T cells were activated for three days (as described above), washed, and plated on Seahorse metabolic flux analysis plates, 5×10⁴ cells per well in a 24-well container. Experiments were done in an XF assay medium that contained 25 mM glucose, 2 mM L-glutamine, and 1 mM sodium pyruvate and analyzed using a Seahorse XF24 extracellular flux analyzer (Agilent Technologies). When indicated, the following were injected: oligomycin (1.5 μM), carbonyl cyanide 4-(trifluoromethoxy)phenylhydrazone (FCCP; 1.5 μM), rotenone (100 nM), and antimycin A (1 μM). Basal extracellular acidification rate (ECAR) and oxygen consumption rate (OCR) reports were generated by Wave Desktop software (Agilent Technologies).

The procedures regarding total RNA extraction and quantitative RT-PCR are as follows. Total RNA was extracted from cells using TRIzol reagent (Invitrogen, Carlsbad, CA, USA). The quantity and quality of extracted RNA were verified using a NanoDrop spectrophotometer (Thermo Fisher Scientific, Waltham, MA, USA) and Agilent 2100 Bioanalyzer (Agilent Technologies, Santa Clara, CA, USA). Quantitative RT-PCR was performed using a first-strand cDNA synthesis kit for RT-PCR (Roche Diagnostics, Basel, Switzerland) and an ABI ViiA7 real-time PCR system (Applied Biosystems, Foster City, CA) using TaqMan gene expression assays. The results are expressed as a threshold cycle (Ct). The expression level of each gene was normalized to that of GAPDH, and the relative mRNA expression data were calculated using the 2^{-ΔΔCt} method. All experimental samples were compared with the control (uninfected control groups) and expressed as an n-fold difference. The RT-PCR data were statistically analyzed by determining the fold change values. The primer information is listed in Table 2.

TABLE 2

| Assay ID: | Gene symbol: |
|---|---|
| Mm00524825_m1 | Tfb1m |
| Mm00553272_m1 | Polrmt |
| Mm00447485_m1 | Tfam |
| Mm01344233_g1 | Sod1 |
| Mm01313000_m1 | Sod2 |
| Mm00516005_m1 | Hmox1 |
| Mm00769566_m1 | Srxn1 |
| Mm01621996_s1 | Prdx1 |
| Mm01223875_m1 | Slc1a4 |
| Mm00436603_m1 | Slc1a5 |
| Mm00506391_m1 | Slc38a1 |
| Mm00628416_m1 | Slc38a2 |
| Mm00515325_g1 | Atf4 |
| Mm00477784_m1 | Nfe2l2 |
| Mm00803785_m1 | Asns |

The procedures regarding gene set enrichment analysis (GSEA) are as follows. Gene set enrichment analysis (GSEA: http://www.broad.mit.edu/GSEA) was performed to explore the biological pathways involved in glutamine deprivation. The built-in C2, C5, and C6 curated gene sets from Molecular Signatures Database (MSigDB 6.0: www.broadinstitute.org/gsea/msigdb) were used in following examples. The statistical significance of GSEA was analyzed using 1000 permutations. Enrichment was compared between control and asparagine deprivation CD8⁺ T cells. A positive enrichment score indicates that the specific molecular signature correlated with the phenotype of control CD8⁺ cells. The resulting pathways are selected using the normalized enrichment score (NES)>1 and nominal p-value<0.05.

The procedures regarding RNA-sequencing (RNA-Seq) are as follows. Total RNA was isolated from in vitro cultured CD8⁺ T cells using TRIzol reagent (Invitrogen, Waltham, MA, USA), according to the manufacturer's protocol. A NanoDrop spectrophotometer (Thermo Fisher Scientific, Waltham, MA, USA) and an Agilent 2100 Bioanalyzer (Agilent Technologies, Santa Clara, CA, USA) were used to evaluate the quantity and integrity of the extracted RNA. The paired-end sequencing approach was used to sequence the samples on an Illumina sequencing platform (Illumina, San Diego, CA, USA). The sequencing data were then mapped to the reference genome (Mus musculus; GRCm38) using CLC Genomics Workbench v9.5 software (CLC bio, Aarhus, Denmark). The reads per kilobase million (RPKM) number was used to calculate gene expression levels. To identify and compare pathways enriched by the differentially expressed genes (DEGs) from the RNA-seq datasets. Chromatin immunoprecipitation assays (ChIP).

The procedures regarding ATAC-seq are as follows. The ATAC-Seq Kit (Catalogue No. 53150, Active Motif North America) was employed for tagmentation and library preparation following the kit procedures. Briefly, the T cell was activated and cultured in standard (Con) or asparagine-free (Asn) medium for 3 days, and 50,000 to 100,000 cells were collected and lysed with 100 μl of ice-cold ATAC Lysis Buffer. Subsequently, the tagmentation and library preparation were performed according to the instructions of the kit. Three biological replicates were conducted. The sequencing was performed on NovaSeq 6000 with Pair-end 150 bp. Raw reads were trimmed by using NGmerge (v.0.3) and aligned to the mm10 using Bowtie2 (v.2.2.4). The peak calling as well as the removal of duplicate reads and mitochondria chromosome, were performed by using Genrich (v.0.6.1). The identified peaks from all samples were merged into a consensus peak list, and the chromatin accessibility of each consensus peak in each sample was quantified by using featureCounts function in R with Rsubread package. The presences of open chromatin were used to perform PCA analysis by R. The differential accessible regions were calculated by R with DESeq2 package. The differentially accessible regions were defined according to P value≤0.05 and |log 2 (fold change)|≥1 for the following transcription factor enrichment analysis. The transcription factor enrichment analysis was performed by i-cisTarget (see Imrichova, H., Hulselmans, G., Atak, Z. K., Potier, D. & Aerts, S. i-cisTarget 2015 update: generalized cis-regulatory enrichment analysis in human, mouse and fly. Nucleic Acids Res 43, W57-64, doi: 10.1093/nar/gkv395 (2015)).

The procedures regarding 18F-FDG PET/CT imaging are as follows. All enrolled patients adhered to a minimum fasting duration of 6 hours prior to undergoing 18F-FDG PET/CT imaging, utilizing a PET/CT system (Discovery ST 16; GE Healthcare, Milwaukee, WI) comprising a PET scanner and a 16-section CT scanner. Preceding PET acquisition, a helical CT scan was performed from the head to the proximal thigh following a standardized protocol with the following parameters: transverse 3.0-mm collimation×16 modes, 100 kVp, 100 mAs, tube rotation: 0.5-s tube rotation, table speed: 35 mm/s, and pitch: 1.5. Intravenous iodinated contrast agents were not administered. The CT data were resized from a 512×512 matrix to a 128×128 matrix to align with PET data, facilitating the fusion of corresponding images and the generation of CT-based transmission maps. Emission scans, covering the region from the head to the proximal thigh, were conducted 50-70 minutes post the injection of 370 MBq of 18F-FDG. Two-dimensional mode acquisition (3 minutes for each table position) was employed. PET images underwent reconstruction using CT data for attenuation correction, utilizing an ordered-subset expectation maximization iterative reconstruction algorithm (four iterations and ten subsets). The nuclear medicine physician reviewed all images and identified lesions characterized by an increased tracer uptake. General Electric (GE) Advanced Workstation (AW) Server software (GE Healthcare, Waukesha, WI, USA) was used for manual delineation of volumes of interest on each lesion. The segmentation of lesions was performed by applying a threshold for voxel standardized uptake values (SUVs) as 3 (g/ml). We calculated the following parameters: TLG (total lesion glycolysis), maximum SUV (SUVmax), Peak SUV (SUVpeak), and metabolic tumor volume (MTV).

The procedures regarding determination of EBV DNA copy number are as follows. The EBV DNA copy number for each patient was determined following previously established procedures (see Le, Q. T. et al. An international collaboration to harmonize the quantitative plasma Epstein-Barr virus DNA assay for future biomarker-guided trials in nasopharyngeal carcinoma. Clin Cancer Res 19, 2208-2215, doi: 10.1158/1078-0432.CCR-12-3702 (2013); Hsu, C. L. et al. Plasma Epstein-Barr virus DNA concentration and clearance rate as novel prognostic factors for metastatic nasopharyngeal carcinoma. Head Neck 34, 1064-1070, doi: 10.1002/hed.21890 (2012)). In brief, plasma samples were acquired by centrifuging venous blood collected in an EDTA-coated tube. The cell-free DNA was extracted using the QIAamp DNA Blood Mini Kit (Qiagen). The EBV DNA copy number was quantified using a real-time quantitative polymerase chain reaction (qPCR) system targeting the BamHI-W fragment region of the EBV genome, employing the Applied Biosystems 7500 qPCR system (Thermo Fisher Scientific). Plasma concentration of EBV DNA (copies/mL) was calculated after calibration with the EBV World Health Organization international standard.

First, this example aimed to delineate the impact of combination therapy on the functions of CD8⁺ tumor-infiltrating lymphocytes (TILs).

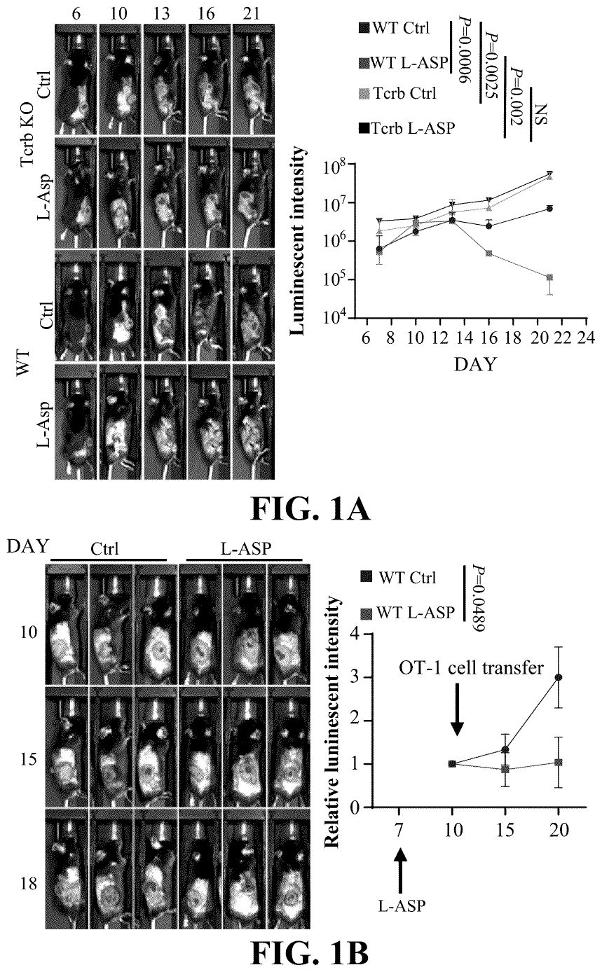

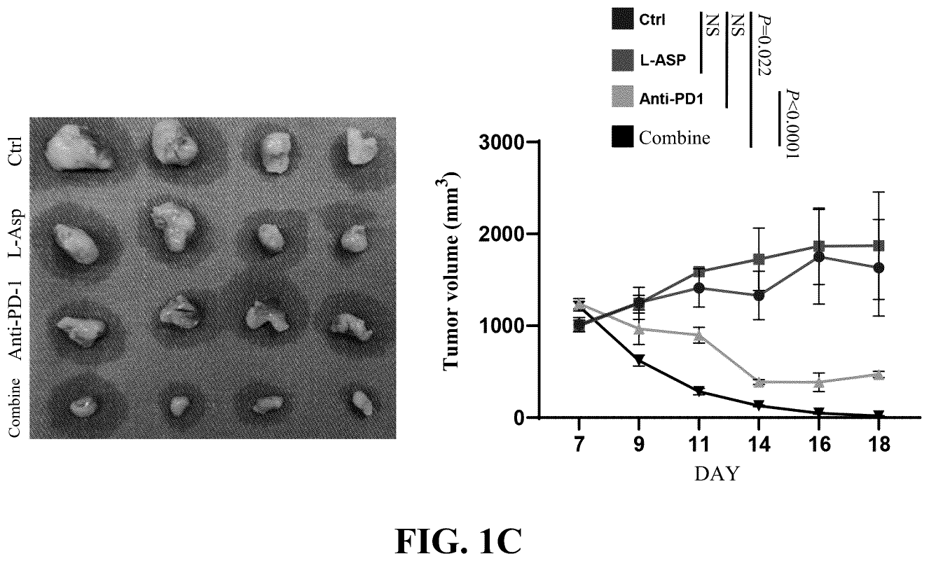

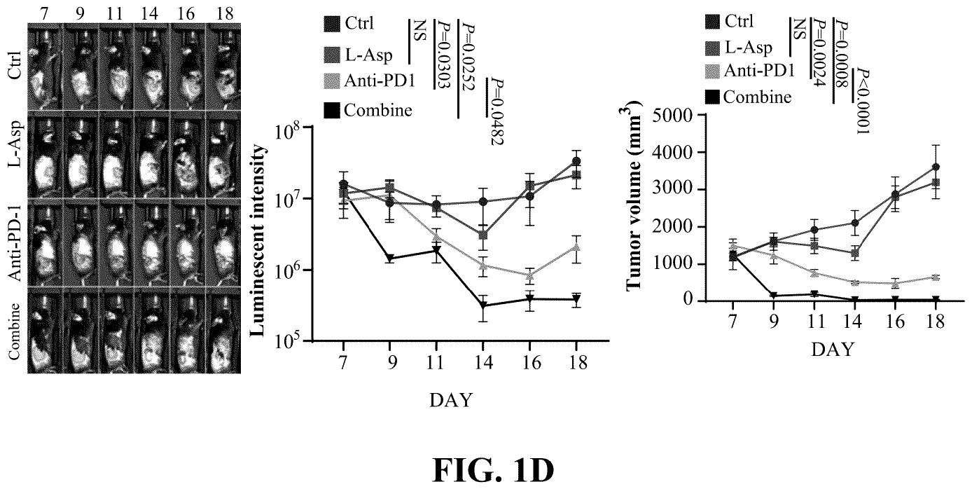

FIGS. 1A-1D show that asparaginase synergize with anti-PD-1 to elevate T cell anti-tumor responses. (1A) B16-F10-Luc-bearing C57BL/6 and Tcrb KO mice were administered with vehicle or L-asparaginase (5 U/g) three days before tumor inoculation and then 3 times per week by intraperitoneal injection. The average luminescent intensity of photons emitted from each tumor in the images was quantified. Data are presented as the mean±SEM (n=4 for each group). (1B) B16-OVA-bearing C57BL/6 mice were administered with vehicle or L-asparaginase (5 U/g) or PBS on day 7 after tumor inoculation and received 1.5×10\hat{ }6 activated OTI T cells on day 10. The average luminescent intensity of photons emitted from each tumor in the images was measured. Error bars represent the standard error of the mean (SEM). Data is representative of one independent experiment with n=3 mice per group. (1C) MTCQ1-bearing C57BL/6 were treated with either a vehicle, L-ASP, anti-PD-1, or a combination of L-ASP and anti-PD-1 starting on day 7 after tumor inoculation. The average tumor volume is emitted from each tumor group. (1D) B16-OVA-bearing C57BL/6 mice were treated with either a vehicle, L-ASP, anti-PD-1, or a combination of L-ASP and anti-PD-1 starting on day 7 after tumor inoculation. The average luminescent intensity of photons and tumor volume are emitted from each tumor group.

We observed that asparaginase treatment led to a robust reduction of tumor burden in wild-type mice but failed to suppress tumor cell growth in TCRb KO mice (see FIG. 1A), highlighting that asparaginase intervention can induce T cell-dependent anti-tumor responses. In support of this, we found that B16-OVA tumor-bearing mice treated with asparaginase prior to transferring of tumor-specific OT-1 CD8⁺ T cells completely impede tumor growth (see FIG. 1B).

We next investigated whether the combination of asparaginase and PD-1 blockade can elicit distinct T cell immune profile and anti-tumor responses compared to single treatment. To better mimic clinical application, we first engrafted MTCQ1 oral carcinoma (OSCC) (see Chen, Y. F., Chang, K. W., Yang, I. T., Tu, H. F. & Lin, S. C. Establishment of syngeneic murine model for oral cancer therapy. Oral Oncol 95, 194-201, doi: 10.1016/j.oraloncology.2019.06.026 (2019)) and B16-F10 melanomas. Our result showed that the combination of asparaginase and anti-PD-1 induced the most potent anti-tumor responses compared to individual treatment in tumour-bearing mice (see FIGS. 1C-1D).

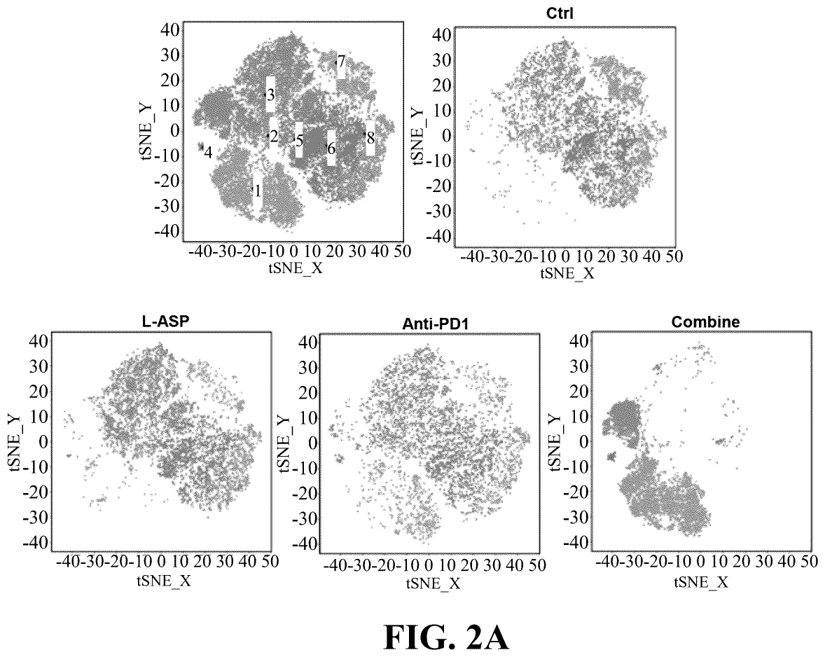

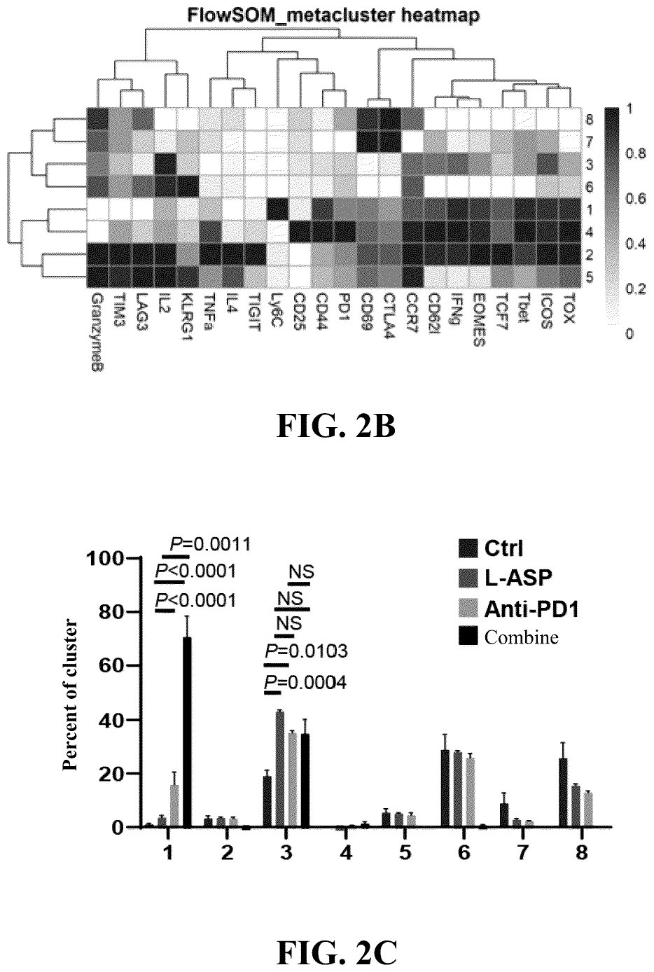

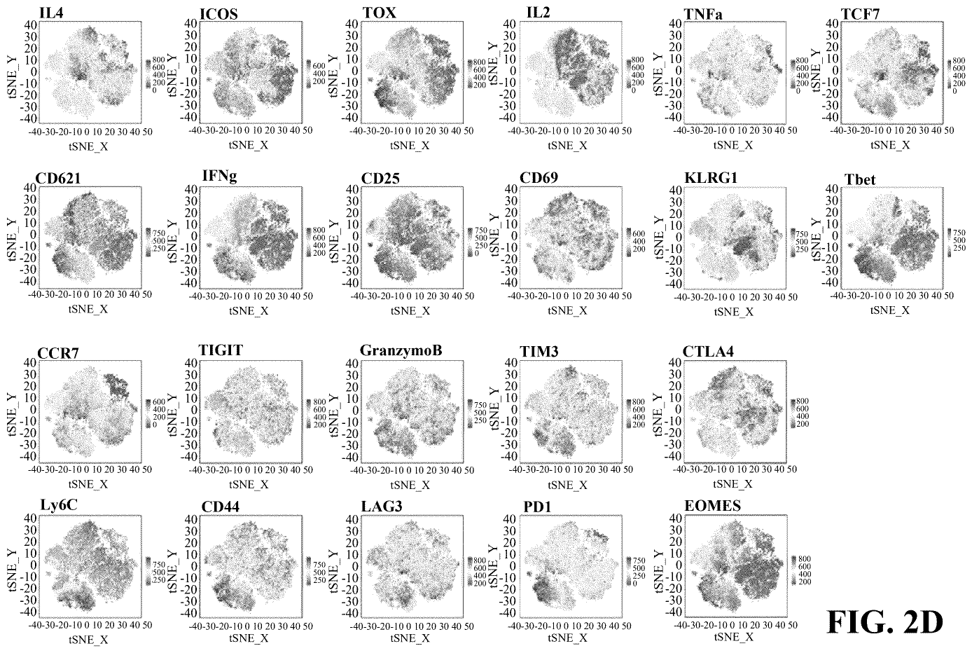

FIGS. 2A-2D show that asparaginase boost PD-1 blockade-induced CD8⁺ T cell activation. (2A) tSNE projection of Tcrb⁺CD45⁺CD8⁺ TIL clusters identified by FlowSOM clustering and percentage of TIL from different groups in each FlowSOM cluster (n=3). (2B) FlowSOM clusters, with a heat map of effector molecule MFIs overlaid onto the t-SNE analysis. (2C) tSNE projection of CD8⁺ T cells for all groups with different treatments. (2D) tSNE projections of indicated protein expression in FIG. 2A. All data are mean±SD and were analyzed by two-tailed unpaired Student's t-test. NS, not significant.

We further analyzed CD8⁺ TILs in those treated mice with FlowSOM clustering (see FIG. 2A). Our result showed that combination therapy led a strong increase in cluster 1, which exhibited increased expression of IFN-γ. CD44 and transcription factors (T-bet and EOMES), suggesting that combination therapy may potentiate CD8 T cell activation in vivo (see FIGS. 2B-2C).

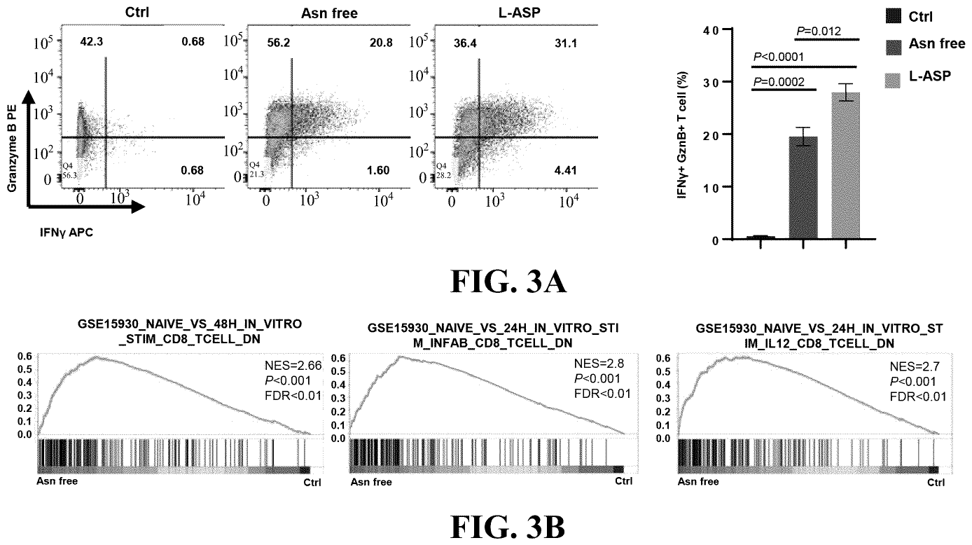

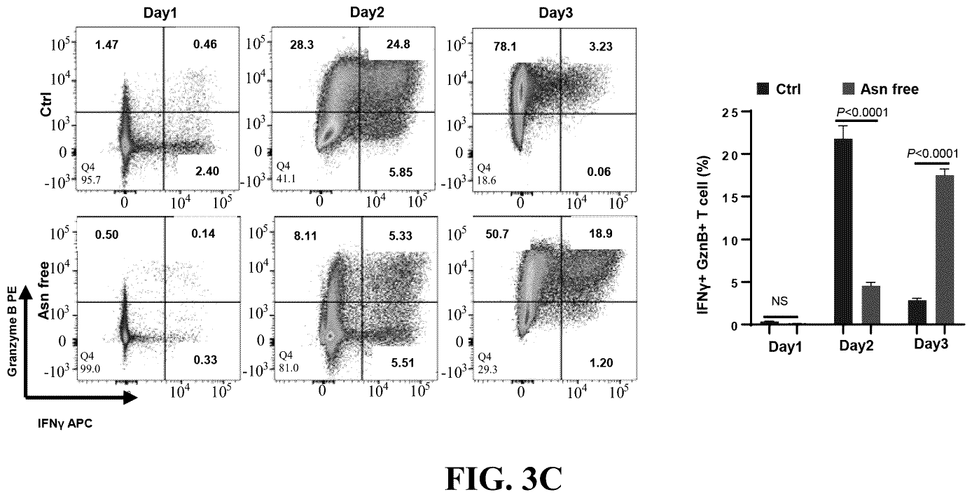

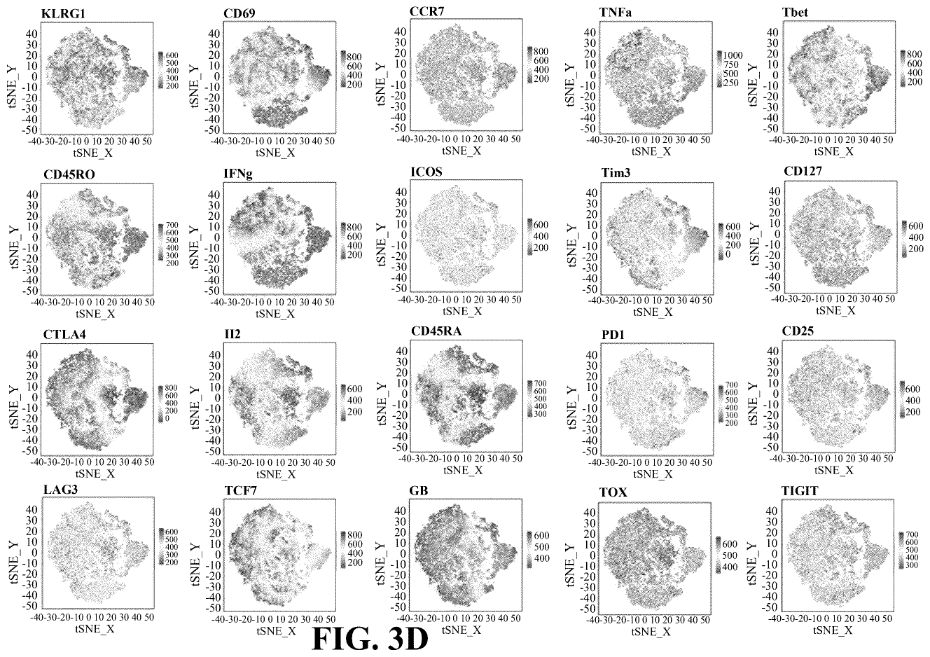

FIGS. 3A-3D show that asparagine deprivation enhances CD8⁺ T cell effector functions. Naïve CD8⁺ T cells were isolated from WT B6 mice and cultured under CD8 differentiation conditions using a medium without asparagine (Asn-free) or with asparaginase (L-ASP). (3A) After three days of culture, the cells were re-stimulated with PMA and ionomycin for four hours in the presence of Golgi stop, permeabilized, and stained for IFN-γ and granzyme B. (3B) GSEA tracing for T cell activation in Asn-free and control cultured CD8⁺ T cell. ES, enrichment score; NES, normalized enrichment score; FDR, false discovery ratio. (3C) human CD8⁺ T cells were isolated from human PBMCs using the Naive CD8⁺ T Cell Isolation Kit, human. CD8⁺ T cells cultured under control and asparagine-deprived conditions. After 3 days cultured, cells were then re-stimulated with PMA and ionomycin for 4 hours with Golgi stop, permeabilized and stained for IFN-γ and granzyme B. (3D) tSNE projections of expression of the indicated proteins. All data are mean±SD and were analyzed by two-way ANOVA with Sidak multiple comparisons tests with adjusted P value and data are cumulative results from at least three independent experiments.

Altogether, our results imply that asparaginase augments the efficacy of anti-PD1 by fostering enhanced activation of immune cells and provide experimental evidence for applying asparaginase and anti-PD-1 combination therapy in better unleashing host anti-tumor immunity. Our results showed that activated murine CD8⁺ T cells from both asparagine deprivation and asparaginase-treated groups displayed a higher percentage of cells expressing IFN-γ and granzyme B (see FIG. 3A). By examining transcriptome in CD8⁺ T cells activated in asparagine-replete or -depleted conditions, our result showed that CD8⁺ T cells displayed an enrichment in gene signature related to T cell activation and responses induced by IL-12 and IFNβ (see FIG. 3B). Similarly, human CD8⁺ T cells activated in the asparagine-deprived media for 3 days also displayed better ability to produce IFNg and granzyme B upon stimulation (see FIG. 3C).

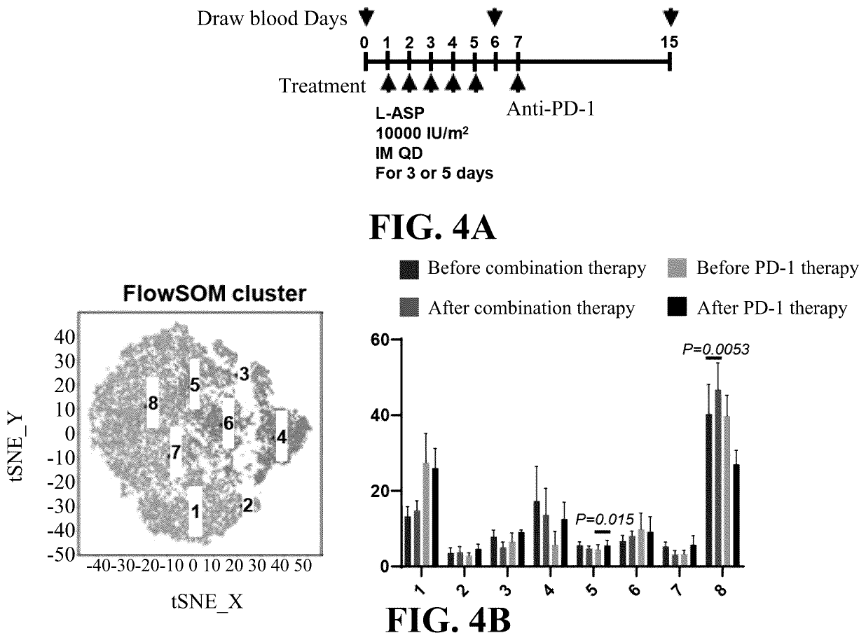

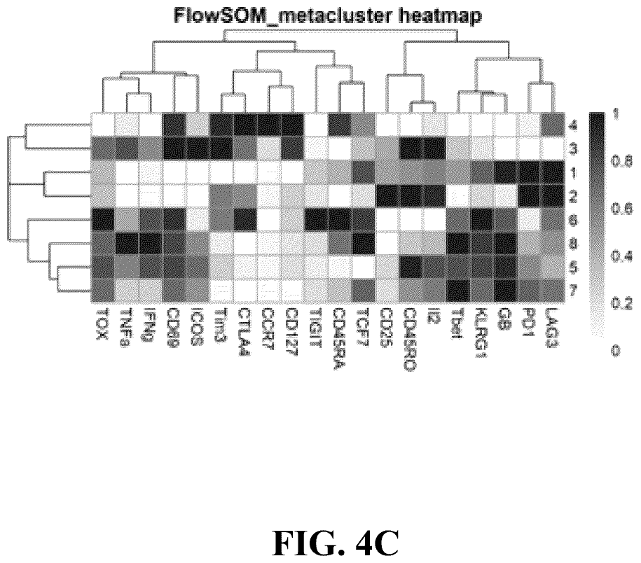

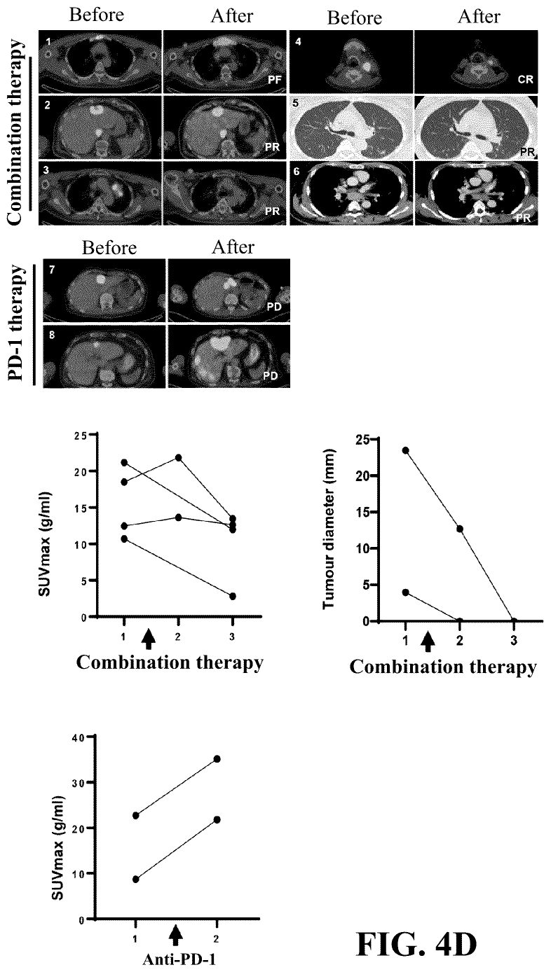

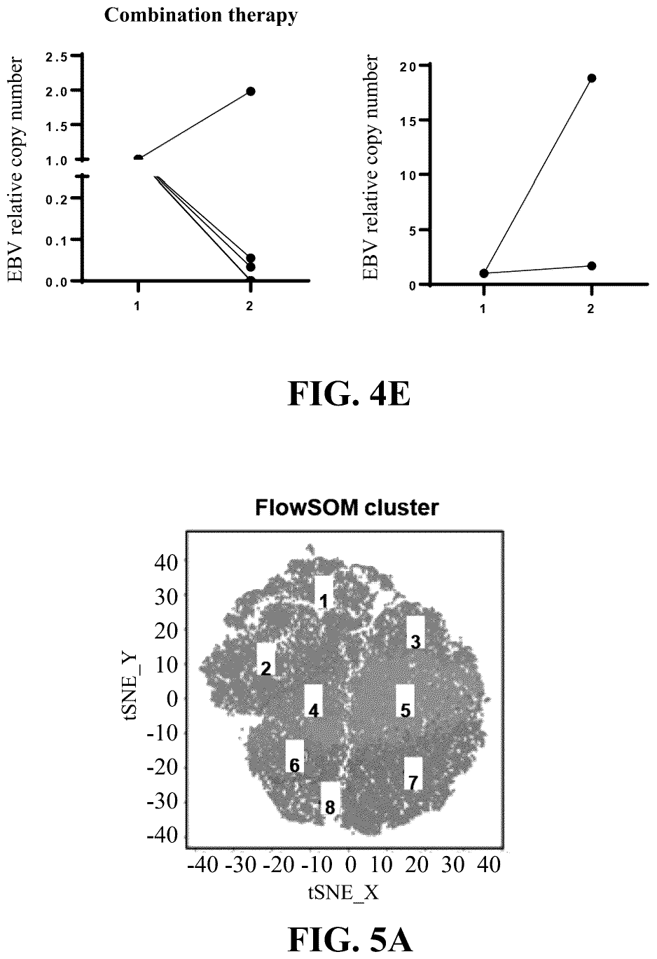

FIGS. 4A-4E show that asparaginase boost PD-1 blockade-induced anti-tumor immunity. (4A) Schematic diagram illustrating the design of a clinical trial for Nasopharyngeal carcinoma (NPC) patients. (4B) t-SNE analysis of CD45⁺CD8⁺ T cells from PBMC populations (n=8) identifies distinct. Percentage of CD45⁺CD8⁺ T cells from different days in each FlowSOM cluster. (4C) FlowSOM clusters, with a heat map of effector molecule MFIs overlaid onto the t-SNE analysis. (4D) PET scans of 18F-FDG in patients with NPC and the maximum standardized uptake value (SUVmax) before and after receiving asparaginase and pembrolizumab combination therapy and (4E) plasma EBV relative DNA copy number distribution in patients with metastatic NPC conduct combination therapy (6 patients) and PD-1 therapy (2 patients). All data are mean±SD and were analyzed by two-tailed, paired Student's t-test. NS, not significant.

For compassionate therapy, we enrolled six RM-NPC patients who have received PD-1 therapy but are still under progressing diseases. They received sequential asparaginase (daily injections for three or five days) followed by pembrolizumab, an anti-PD-1 mAb (single injection), and compared the results to a control group receiving only the anti-PD-1 mAb (see FIG. 4A). We performed FlowSOM (see FIG. 4B and FIG. 3D) to better define the changes induced by treatments. We found that combination therapy led to a notable increase in Clusters 8 (FIG. 4C), reflecting populations with heightened cytokine production and elevated activation markers. Concurrently, Cluster 6 expressing lower cytokines and some inhibitory receptors also increased, suggesting an augmented population of effector and exhausted T cells post combination therapy. In the PD-1 group, cluster 5 expressing cytokines and activation have slightly elevated. In addition to profiling CD8⁺ T cells, we performed a positron emission tomography-computed tomography (PET/CT) imaging and found that combination therapy declined the SUV signal and two of the patients had decreased tumor volume (see FIG. 4D). Most importantly, the EBV DNA copy number, which is correlated with NPC burden, showed a robust decline after the combination treatment but not after PD-1 treatment alone (see FIG. 4E), indicating that the combination therapy effectively altered T cell activation and suppressed tumor progression. Here, we found that asparaginase combined with the anti-PD-1 antibody elicited robust anti-tumor responses in NPC patients accompanied with inflammatory signs. By combining multi-dimensional flow analysis in both NPC patients and tumor-bearing murine models, we unveiled that asparaginase treatment modulated CD8⁺ T cell differentiation state and stimulated T cell-dependent anti-tumor immunity in combination with the anti-PD-1 antibody.

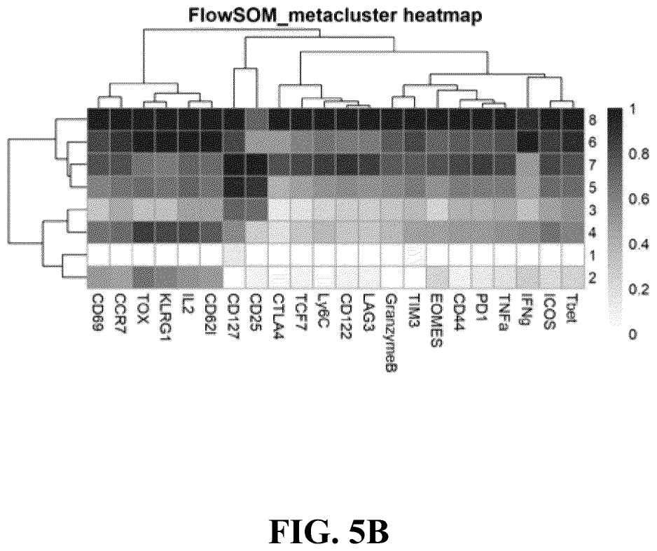

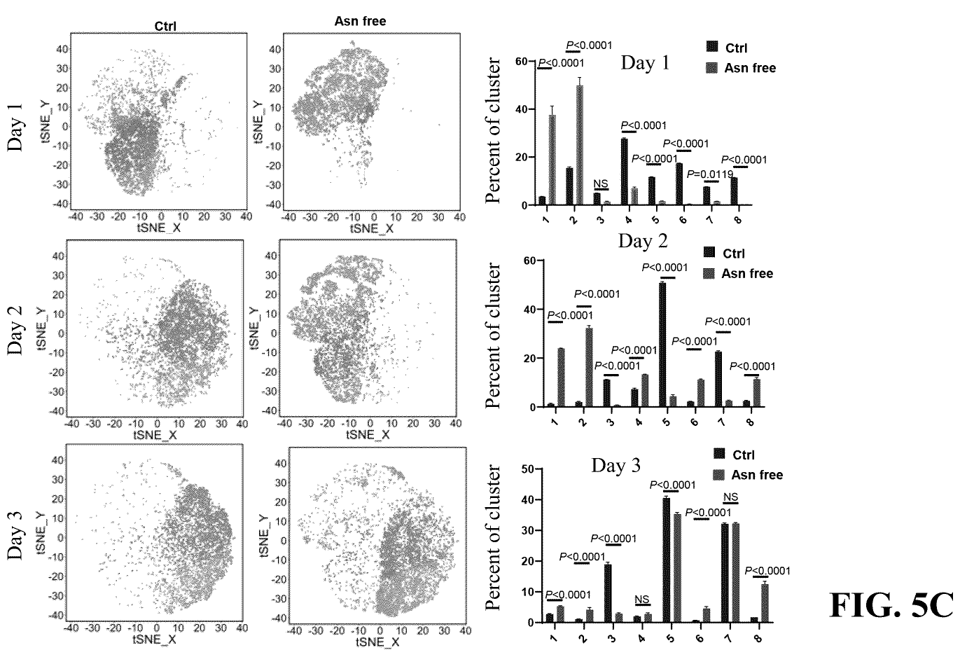

FIGS. 5A-5C show that asparagine deprivation enhances CD8⁺ T cell effector functions. Naïve CD8⁺ T cells were isolated from WT B6 mice and cultured under CD8 differentiation conditions using a medium without asparagine (Asn-free). For various durations, the cells were re-stimulated with PMA and ionomycin for four hours in the presence of Golgi stop, permeabilized, and stained for IFN-γ and granzyme B. (5A) t-SNE analysis of CD8⁺ T cells in differentiation conditions using both control medium and asparagine-deprived medium (5B) with a heat map of effector molecule MFIs overlaid onto the t-SNE analysis. (5C) tSNE projection of CD8⁺ T cells for all groups with various durations and percentages of CD8⁺ T cells from different days in each FlowSOM cluster (n=3). All data are mean±SD and were analyzed by two-way ANOVA with Sidak multiple comparisons tests with adjusted P value and data are cumulative results from at least three independent experiments.

To gain a comprehensive understanding of the impact of asparagine deprivation on T cell activation, we cultured CD8⁺ T cells in vitro in asparagine-replete and -depleted condition over 3 days and performed FlowSOM clustering (see FIGS. 5A-5C). We found that asparagine deprivation could initially induce delayed T cell activation, but a potential adaptation process induced by prolonged asparagine deprivation eventually potentiates T cell differentiation toward populations with more superior effector functions.

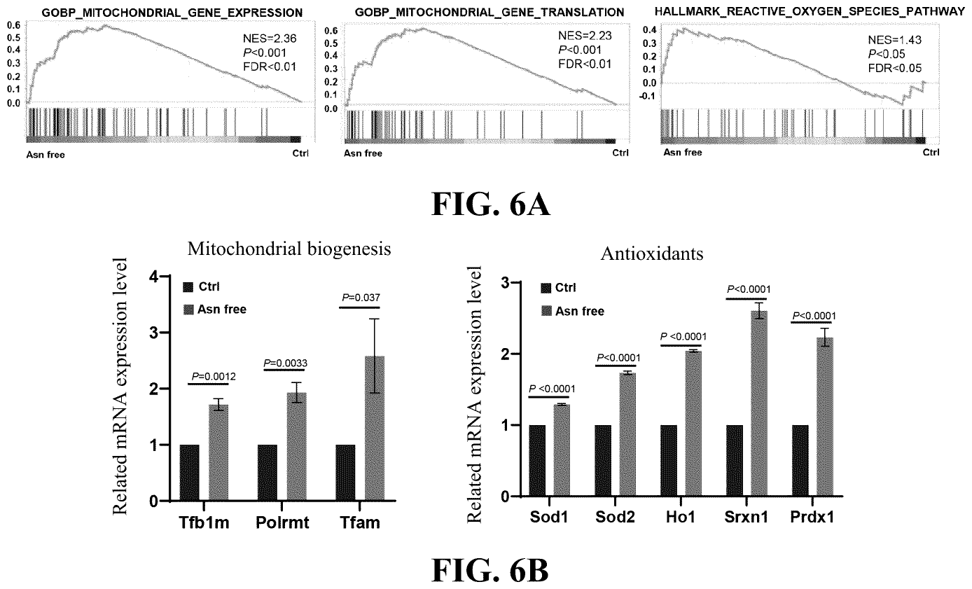

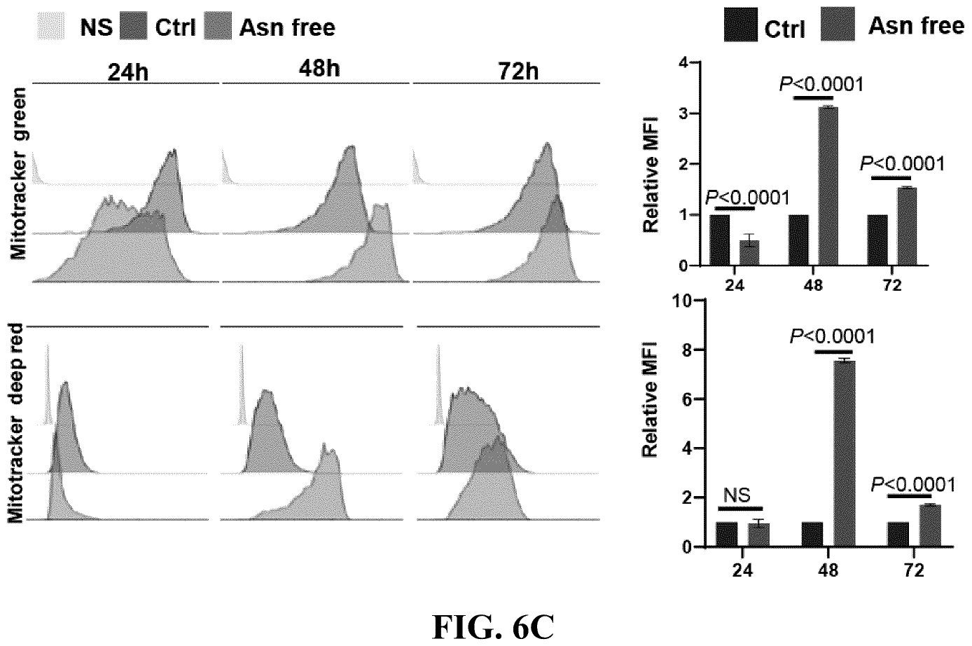

FIGS. 6A-6C show that asparagine deprivation reprograms mitochondrial fitness in T cells. (6A) GSEA tracing for mitochondrial biogenesis in Asn-free and control cultured CD8⁺ T cell. ES, enrichment score: NES, normalized enrichment score; FDR, false discovery ratio. (6B) The level of indicated mRNAs in control T cells and asparagine-free T cell were determined by qPCR. (6C) Naïve CD8⁺ T cells cultured in asparagine-free and control medium for several days and stained with MitoTracker™ Green FM Dye and MitoTracker™, Deep Red FM Dye. Student's t-test or two-way ANOVA with Sidak multiple comparisons tests with adjusted P value (6B, 6C) and data are cumulative results from at least three independent experiments. NS, not significant.

By RNA seq analysis, we first found that T cells activated in the absence of asparagine elevated gene signatures related to elevated mitochondrial activity (see FIGS. 6A-6B). Our result showed that asparagine deprivation for 24h suppressed mitochondrial mass and membrane potential; however, there were robust increases in both mitochondrial mass and membrane potential in T cells cultured in asparagine-deprived condition for 48 h and 72h (see FIG. 6C). The results indicate that prolonged asparagine deprivation can induce opposite outcomes in mitochondrial state as well as T cell differentiation pattern, which potentially result from adaptation induced by prolonged asparagine deprivation.

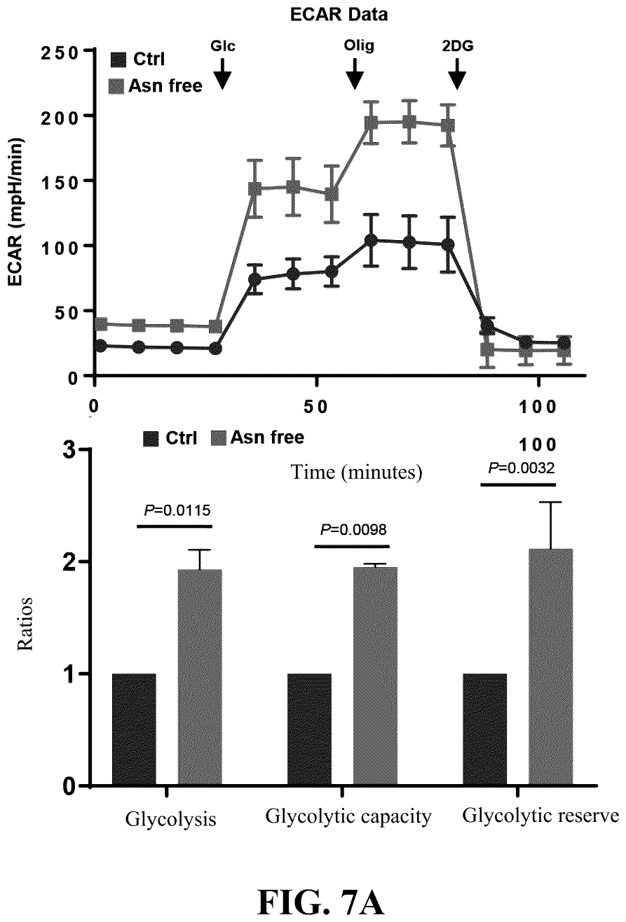

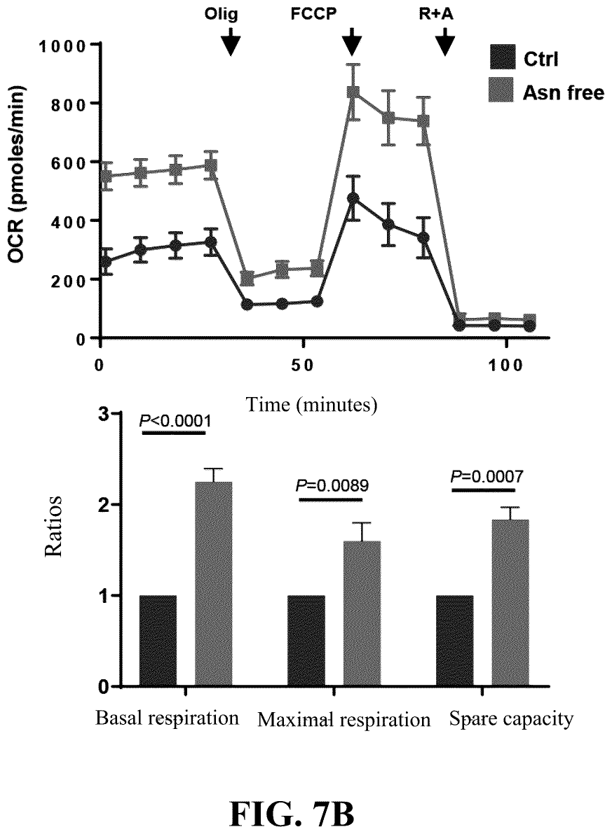

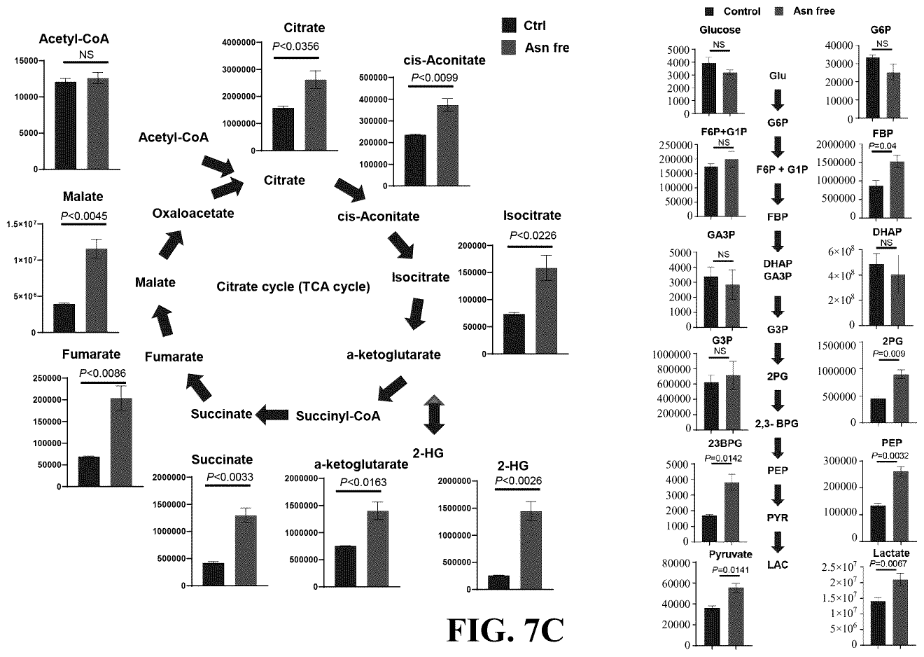

FIGS. 7A-7C show that asparagine deprivation influences metabolic preferences in T cells. (7A) Traces of oxygen consumption rates (OCRs) were recorded. The following compounds were injected into the assay micro-chambers as indicated: oligomycin (Olig.), carbonyl cyanide-4-(trifluoromethoxy)phenylhydrazone (FCCP), and rotenone plus antimycin A (R+A). (7B) Traces of extracellular acidification rates (ECARs) were recorded. Compounds injected included glucose (Glc), oligomycin, and 2-deoxy glucose (2DG). Each data point represents the mean±standard deviation (SD) of four technical replicates. (7C) Lysed cells from a control medium or an asparagine-deprived medium 3-day cultured CD8⁺ T cell. The cells were subsequently analyzed using liquid chromatography-mass spectrometry (LC-MS) to assess the glycolysis (right) and TCA cycle (left). Student's t-test or two-way ANOVA with Sidak multiple comparisons tests with adjusted P value and data are cumulative results from at least three independent experiments. NS, not significant.

To further investigate whether asparagine deprivation affect T cell metabolic program, we perform the seahorse extracellular flux assay (see FIGS. 7A-7B) and targeted metabolomics (see FIG. 7C). The data revealed that asparagine deprivation promoted bioenergetic programs, including OXPHOS and glycolysis, in T cells.

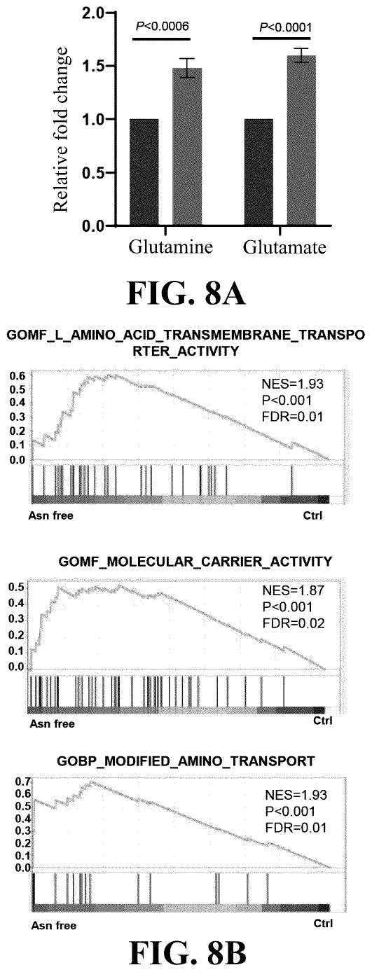

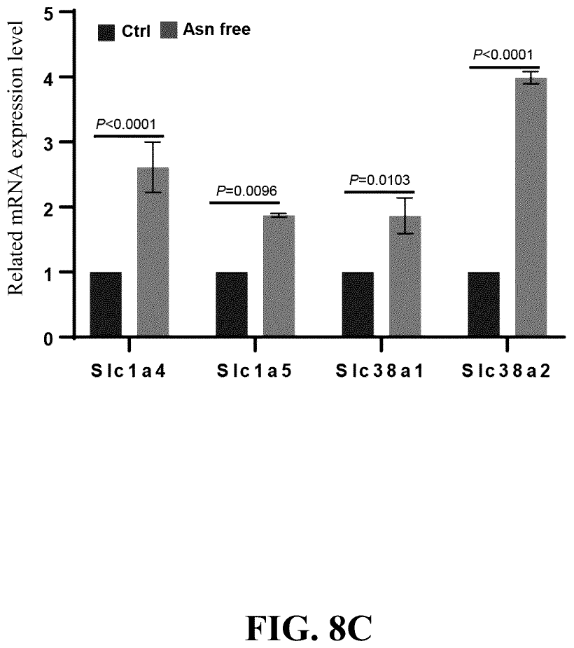

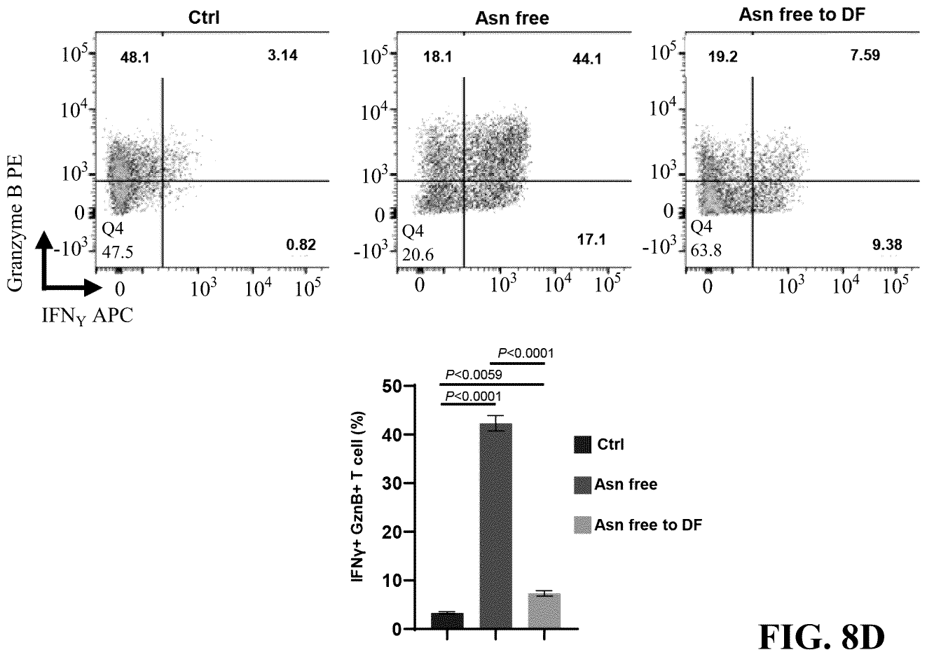

FIGS. 8A-8D show that asparagine deprivation affects glutamine metabolism to enhance CD8⁺ T cell activation. (8A) Lysed cells from a control medium or an asparagine-deprived medium 3-day cultured CD8⁺ T cell. The cells were subsequently analyzed using liquid chromatography-mass spectrometry (LC-MS) to assess the glutamine and glutamate. (8B) GSEA tracing for mitochondrial biogenesis in Asn-free and control cultured CD8⁺ T cell. ES, enrichment score; NES, normalized enrichment score; FDR, false discovery ratio. (8C) The level of indicated mRNAs in control T cells and asparagine-free T cell were determined by qPCR. (8D) Naïve CD8⁺ T cells were cultured in CD8 differentiation conditions with either asparagine-free or control medium for three days. For the Asn-free to DF condition (Asparagine and Glutamine-free), after two days of culture in an asparagine-free medium, the cells were transferred to a medium lacking both asparagine and glutamine for one day and then stained for IFN-γ and granzyme B by flow cytometry. All data are mean±SD and were analyzed by two-tailed paired Student's t-test or two-way ANOVA with Sidak multiple comparisons tests with adjusted P value and data are cumulative results from at least three independent experiments.

Intriguingly, we found that T cells cultured in the asparagine-depleted condition increased glutamine and glutamate levels (see FIG. 8A) and enriched in the gene set involved in amino acid transporter activity and critical glutamine transporters (see FIGS. 8B-8C), indicating that asparagine-deprived T cells may display a potential enhancement in glutamine metabolism. To explore whether the enhancement of glutamine metabolism is required to promote effector functions in T cells upon asparagine deprivation, we activated T cells in the asparagine-replete or -depleted media for two days. T cells activated in the absence of asparagine were then switched to either asparagine-depleted media or media deprived with both asparagine and glutamine for one more day. Our result showed that glutamine deprivation robustly abolished the enhancement of effector cytokine production in asparagine-deprived T cells (see FIG. 8D), indicating that glutamine plays a critical to tailor effector function in response to asparagine deprivation in CD8⁺ T cells. Altogether, our results highlight that asparagine deprivation rewires metabolic program in a stage-dependent manner and the elevated glutamine metabolism plays a crucial role in enabling CD8⁺ T cells to mount elevated effector function in response to asparagine deprivation.

To further elucidate how asparagine deprivation modulate differentiation and effector function of CD8⁺ T cells, we performed Assay for Transposase-Accessible Chromatin with high-throughput sequencing (ATAC-seq) for CD8⁺ T cell activated in control or asparagine-depleted condition for 3 days.

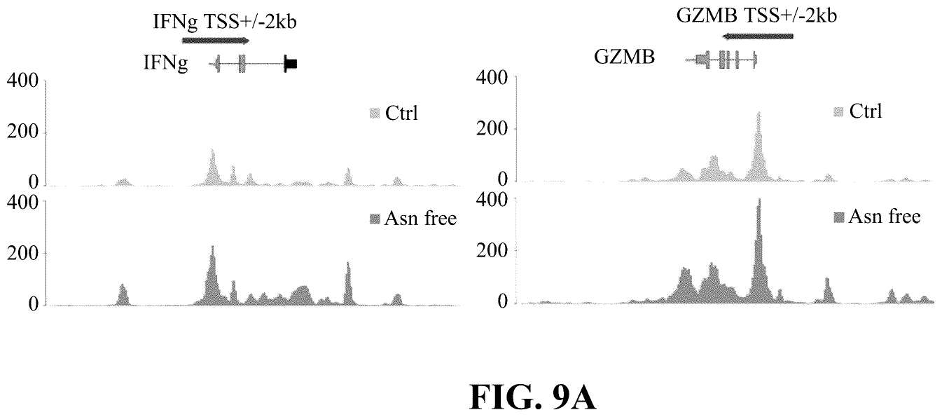

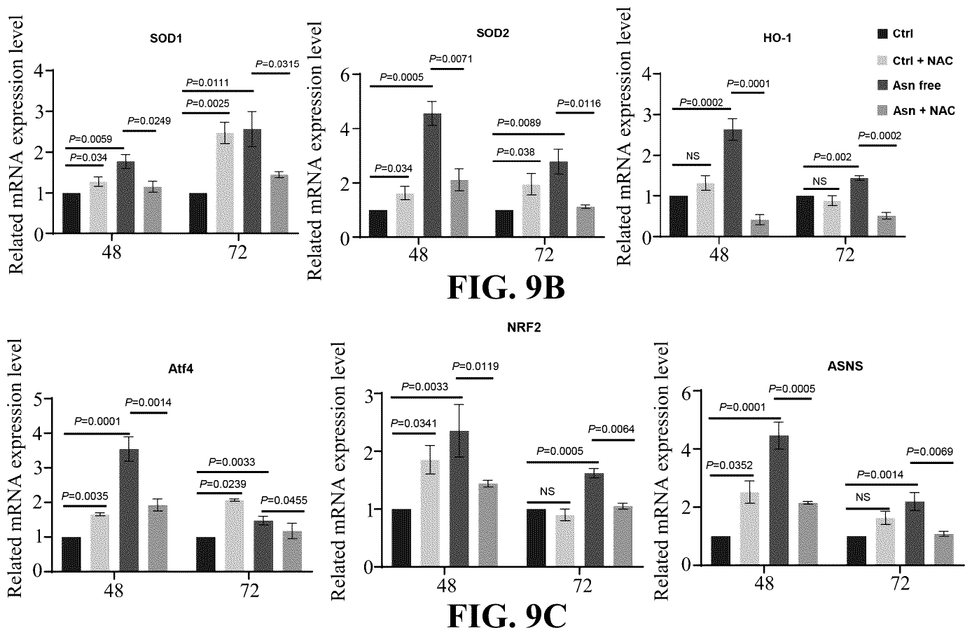

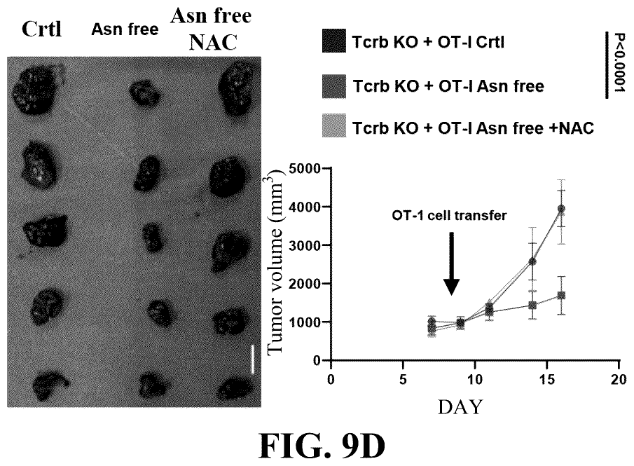

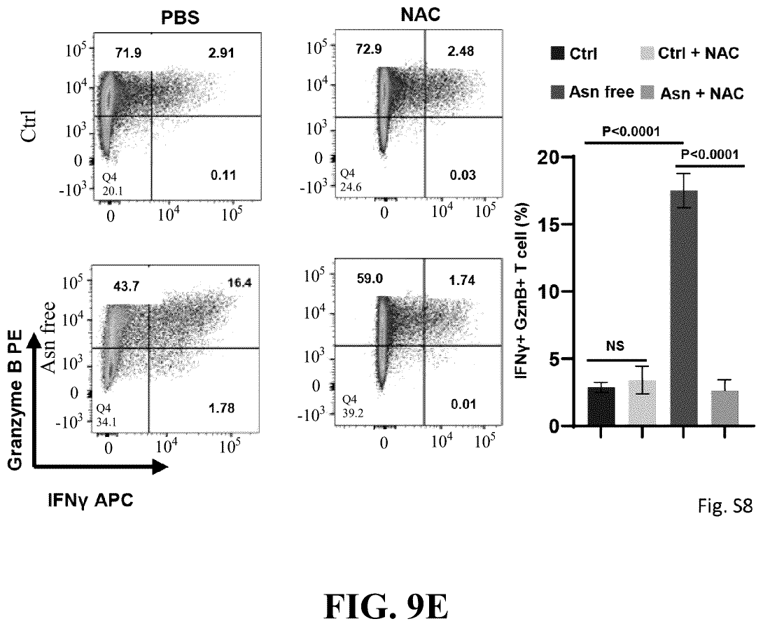

FIGS. 9A-9E show that asparagine deprivations modulates chromatin accessibility pattern in T cells. (9A) The signal of open chromatin in both Asn-free and control conditions on Day 3 are represented in the genomics locus of IFNG and GZMB. The orange indicated the Asn-free condition whereas the blue indicated the control condition. (9B, 9C) The level of indicated mRNAs in T cells cultured under control and asparagine-deprived conditions in the presence of N-acetylcysteine (NAC) were determined by qPCR. (9D) B16-OVA-bearing Tcrb KO mice were received 1.5×10\hat{ }6 activated OTI T cells on day 7. Data are presented as the mean±SEM (n=5 for each group). (9E) human CD8⁺ T cells were isolated from human PBMCs using the Naive CD8⁺ T Cell Isolation Kit, human. CD8⁺ T cells cultured under control and asparagine-deprived conditions in the presence of N-acetylcysteine (NAC) for 3 days. Student's t-test and data are cumulative results from at least three independent experiments. NS, not significant.

In line with elevated effector functions, we found that asparagine-deprived T cells displayed higher chromatin accessibility in gene loci, including IFNg and GzmB, compared to control group (see FIG. 9A).

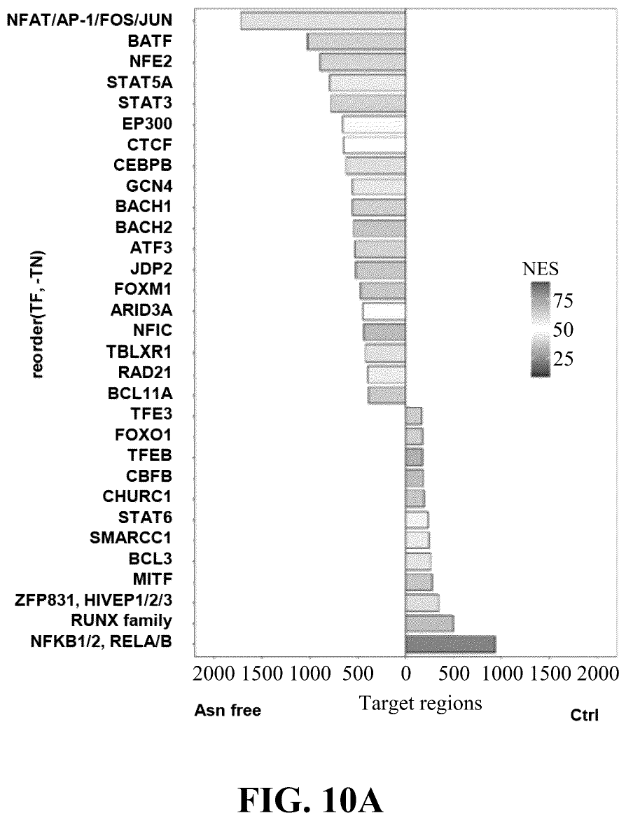

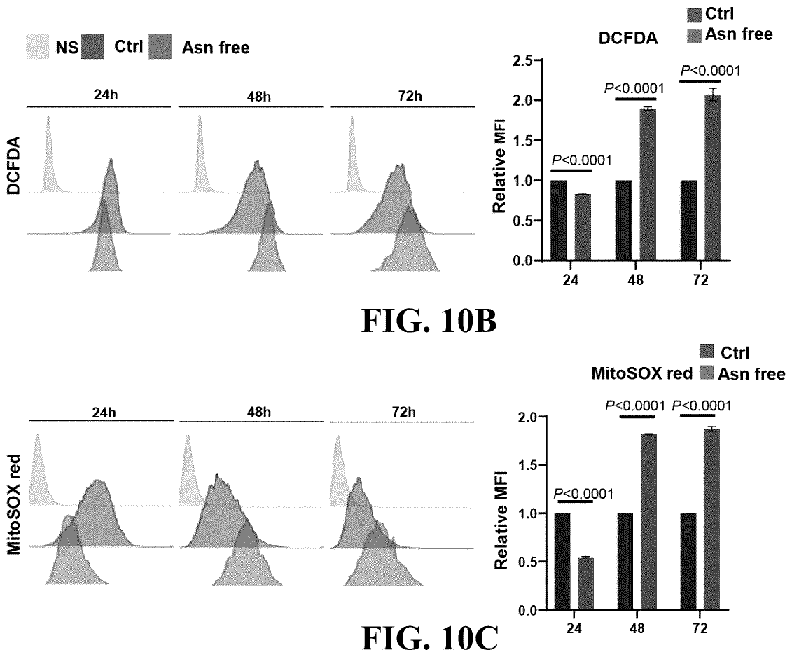

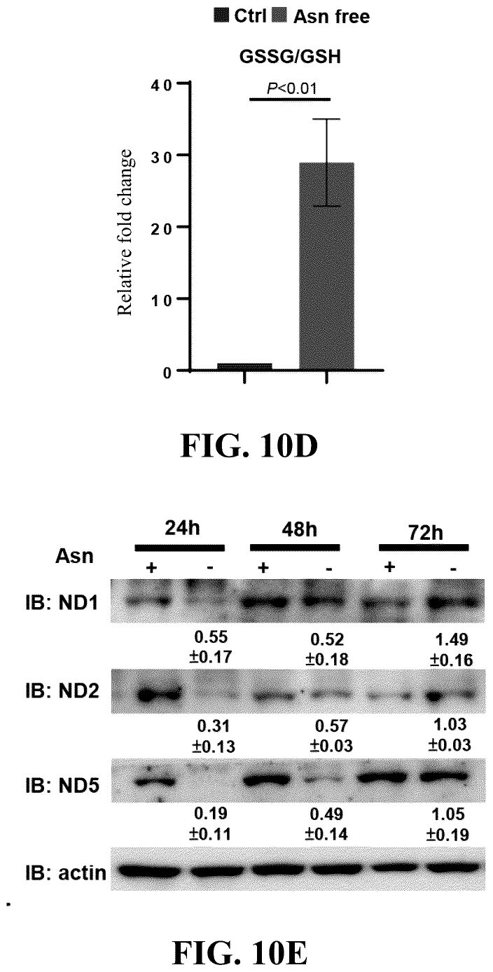

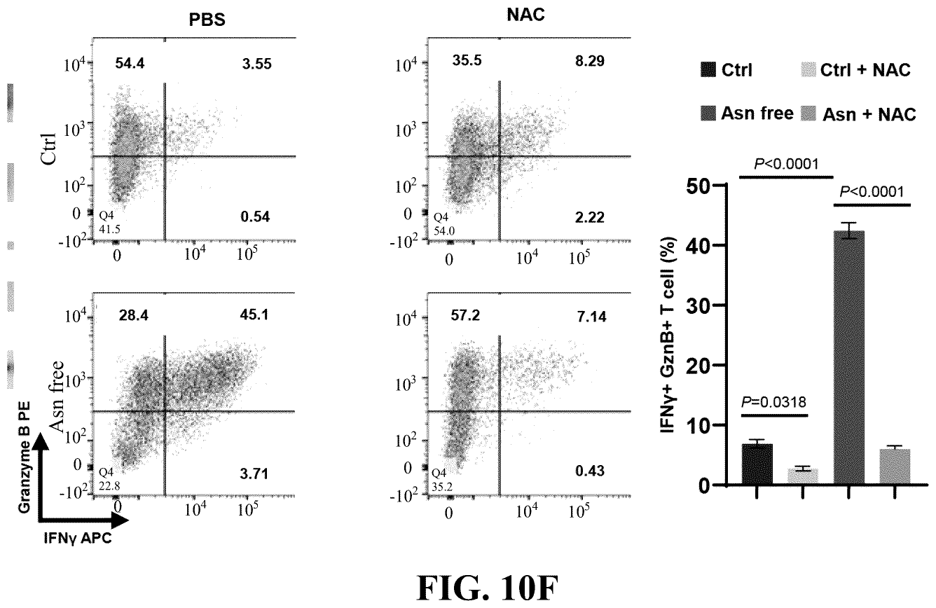

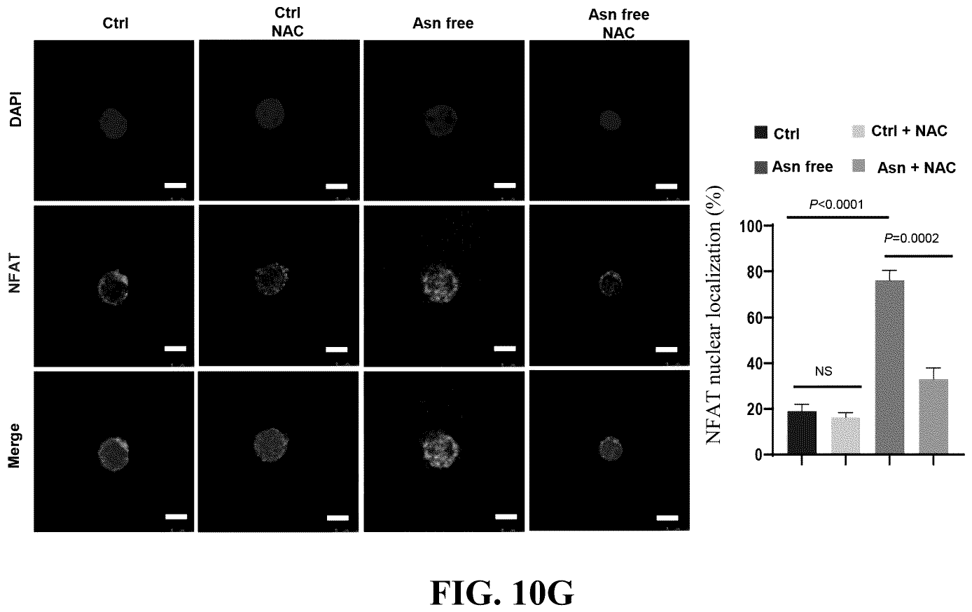

FIGS. 10A-10G show that asparagine deprivation tailors differentiation and metabolic adaptation in a ROS-dependent manner. (10A) The enriched TFs in both Asn-free and control conditions are selected, and the TFs that have the most target region enrichment in either Asn-free or control are represented as the preference. (10B, 10C) Naïve CD8⁺ T cells were cultured under CD8 differentiation conditions in both control medium and asparagine-deprived medium for various durations. Subsequently, the cells were stained for DCFDA and MitoSOX. (10D) Lysed cells from a control medium or an asparagine-deprived medium 3-day cultured CD8⁺ T cell. The cells were subsequently analyzed using liquid chromatography-mass spectrometry (LC-MS) to assess GSSG/GSH ratio. (10E) Naïve CD8⁺ T cells were cultured under CD8 differentiation conditions in both control medium and asparagine-deprived medium for various durations. The protein levels of the indicated molecules were determined through immunoblot analysis. (10F) Naïve CD8⁺ T cells cultured in CD8 differentiation conditions with either asparagine-free or control medium and added with 20 mM NAC for three days, then stained for IFN-γ and granzyme B by flow cytometry. (10G) Imaging of NFAT localization staining (in green) and DAPI (in blue) by confocal microscopy. All data are mean±SD and were analyzed by two-tailed paired (10D) Student's t-test, one-way ANOVA with two-way ANOVA with Tukey's multiple comparisons test (10F, 10G) or Sidak multiple comparisons tests with adjusted P value (10B, 10C) and data are cumulative results from at least three independent experiments.

We next found that the genomic regions more accessible in asparagine-deprived T cells exhibited enrichment in the consensus binding motifs for nuclear factor of activated T cells (NFAT) and basic leucine zipper transcription factor ATF-like (BATF) (see FIG. 10A), two critical transcription factors controlling activation and effector differentiation of T cells. This result highlights that asparagine deprivation may enhance NFAT-mediated transcriptional regulation to support effector T cell differentiation. Since that persistent production of reactive oxygen species (ROS) has been shown to stimulate NFAT signaling and glutamine can modulate redox balance by serving as a precursor for glutathione (GSH), we then postulated that asparagine deprivation may boost ROS production and the elevated glutamine levels may be an adaptation process to protect cells from excessive damage caused by ROS production. In support of this, we indeed observed that T cells from asparagine deprived condition elevated gene signature related to reactive oxygen species pathway (see FIG. 9B). Thus, we examined total ROS and mitochondrial ROS (mROS) in CD8⁺ T cells activated in asparagine-replete and -depleted condition by DCFDA and MitoSOX, respectively. Our result showed that asparaginase deprivation initially suppressed ROS and mROS in the 24h culture; however, prolonged deprivation boosted production of ROS and mROS after 48h (see FIGS. 10B-10C), which is in line with the delayed activation but enhanced effector functions as we observed in our previous results. Furthermore, we found that asparagine deprivation robustly increased glutathione disulfide (GSSG) to GSH ratios in CD8⁺ T cells (see FIG. 10D), indicating that asparagine deprivation promotes production of ROS and consumption of GSH due to antioxidant responses.

Intriguingly, asparagine is a critical amino acid required for the synthesis of essential proteins and mitochondrial genome-encoded proteins (MEPs), including NADH-ubiquinone oxidoreductase chain 1, 2, and 5 (ND1, ND2 and ND5). Interestingly, the deficiency of ND protein subunits has been shown to disturb the activity of electron transport chain and lead to elevated mROS production. We thus examined whether asparagine deprivation could modulate the expression of ND proteins and found that asparagine depletion robustly suppressed ND1. ND2 and ND5 protein expression (see FIG. 10E). However, the expression of ND1, ND2 and ND5 was restored at 72 h post asparagine deprivation. This result suggests that the impaired expression of ND proteins in response to asparagine deprivation may support ROS production and the adaptation process induced by prolonged asparagine deprivation can restore ND protein expression. Interestingly, asparagine restriction has been shown to promote ROS production and activate integrated stress responses, especially activating transcription factor 4 (ATF4), in multiple cell types. Moreover, the elevated ATF4 activity induced by asparagine restriction can stimulate the expression of asparagine synthetase (ASNS) to adapt metabolic stress. We therefore speculated that elevated ROS can promote metabolic adaptation, including ATF4 activation, the expression of ASNS and antioxidant responses, to couple stress responses induced by asparagine restriction. To examine this, we activated T cells with a potent antioxidant, N-acetylcysteine (NAC), in the asparagine-replete or -depleted conditions. Our result showed that asparagine restriction induced expression of ATF4, ASNS and NRF2 (a master transcription factor controlling oxidant signaling pathways) in a NAC-sensitive manner (see FIG. 9C), suggesting that ROS controls metabolic adaptation in response to asparagine restriction. Moreover, we found that NAC treatment abolished asparagine deprivation-mediated enhancement of effector functions in both murine and human CD8⁺ T cells (see FIG. 10F and FIG. 9D). In addition, our result showed that asparagine deprivation can promote NFAT nuclear translocation (an indication of NFAT activation), but the co-treatment of NAC impaired the enhancement of nuclear NFAT levels (see FIG. 10G). Altogether, We delineated that acute asparagine restriction delayed T cell activation, but prolonged asparagine deprivation promoted effector functions in T cells. Mechanistically, prolonged asparagine deprivation led to deregulated expression of mitochondrial complex I subunits, which fostered ROS production and glutamine metabolism to tailor ROS-guided NFAT nuclear localization and activity. As a result of this metabolic adaptation induced by prolonged asparagine restriction, ROS orchestrated distinct T cell differentiation process and promoted effector function in CD8⁺ T cells. We further discovered the bi-phased regulations induced by asparagine deprivation and delineated the underlying metabolic adaptation that tailors T cell differentiation and effector functions. Most importantly, our findings provide proof-of-concept evidence on exploiting asparaginase, a FDA approved chemotherapy in acute lymphoblastic leukemia (ALL) patients, with immune checkpoint inhibitors (ICIs) on treating patients with solid tumors.

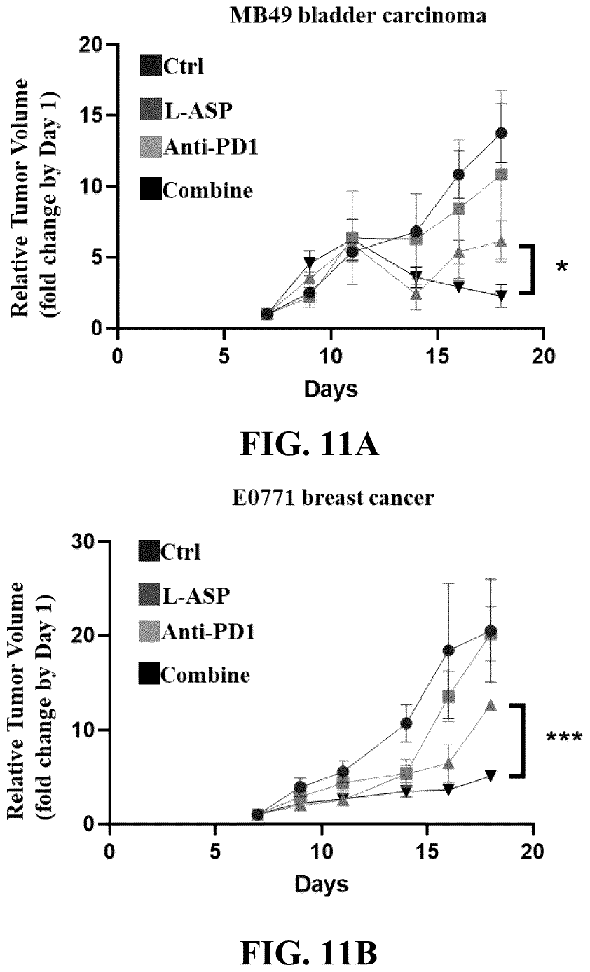

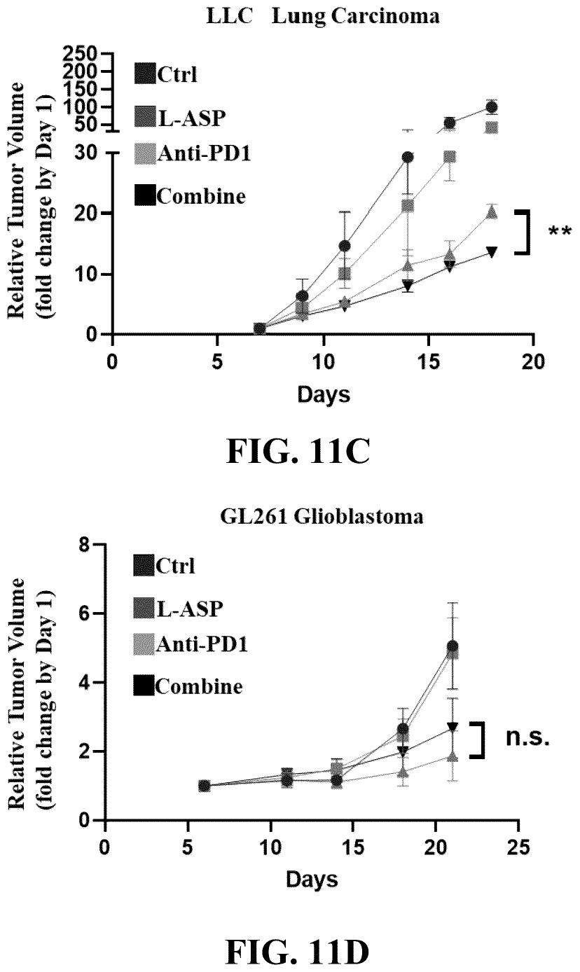

FIGS. 11A-11D show that the synergistic effect of asparaginase and anti-PD-1 in specific tumor type. (11A) MB49 bladder carcinoma (11B) E0771 breast cancer (11C) LLC1 lung carcinoma (11D) GL261 glioblastoma-bearing C57BL/6 were treated with either a vehicle, L-ASP, anti-PD-1, or a combination of L-ASP and anti-PD-1 starting on day 7 after tumor inoculation. The average tumor volume is emitted from each tumor group.

In summary, the results of the present invention are illustrated by the above examples. The present invention discovered that asparagine inhibitory agent can activate cytotoxic T cells, promote the killing effect of cytotoxic T cells on tumors. Clinical and animal experiments have confirmed that asparagine inhibitory agent combined with immune checkpoint inhibitors can enhance the efficacy of cancer immunotherapy by activating killer T cells. The invention proposes a combination therapy using asparagine inhibitory agent and immune checkpoint inhibitors, providing a new strategy for cancer immunotherapy development.

The combination of asparaginase and anti-PD-1 monoclonal antibody (mAb) has shown synergistic anti-tumor effects in murine tumor models. In compassionate therapy for recurrent-metastatic nasopharyngeal carcinoma (RM-NPC) patients, the present invention observed that the combined treatment with L-asparaginase and PD-I blockade mAb induced a robust anti-tumor response dependent on CD8⁺ T cell-mediated immune activity. Further investigation revealed that prolonged asparagine deprivation dysregulates mitochondrial complex I subunit expression, promoting reactive oxygen species (ROS) production and enhancing glutamine metabolism. This ultimately guides nuclear factor of activated T-cells (NFAT) nuclear localization and activity via ROS signaling.

The present invention found that asparaginase combined with the anti-PD-1 antibody elicited robust anti-tumor responses in NPC patients accompanied with inflammatory signs. By combining multi-dimensional flow analysis in both NPC patients and tumor-bearing murine models, we unveiled that asparaginase treatment modulated CD8⁺ T cell differentiation state and stimulated T cell-dependent anti-tumor immunity in combination with the anti-PD-1 antibody.

The present invention can provide a new combination strategy for cancer immunotherapy in clinical medicine, using the combination therapy of an asparagine inhibitor and an immune checkpoint inhibitor to improve the success rate of cancer immunotherapy.

The L-asparaginase drug Leunase® is a clinically used drug for the treatment of leukemia. Through the combined treatment strategy of the present invention, asparaginase can be used in the treatment of cancer patients with solid tumors, such as nasopharyngeal carcinoma, lung cancer, colorectal cancer, liver cancer, urothelial carcinoma, pancreatic cancer, renal cell carcinoma, prostate cancer, etc.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for treating cancer, comprising administering to a subject in need thereof a pharmaceutical composition comprising an effective amount of a combination of an asparagine inhibitor and an immune checkpoint inhibitor (ICI).

2. The method according to claim 1, wherein the cancer is bladder carcinoma, breast cancer, glioblastoma, nasopharyngeal carcinoma, lung cancer, colorectal cancer, liver cancer, urothelial cancer, pancreatic cancer, renal cell carcinoma, prostate cancer, oral cancer, or melanoma.

3. The method according to claim 2, wherein the nasopharyngeal carcinoma is recurrent-metastatic nasopharyngeal carcinoma (RM-NPC).

4. The method according to claim 1, wherein the asparagine inhibitor is L-asparaginase.

5. The method according to claim 1, wherein the immune checkpoint inhibitor is an anti-PD-1 monoclonal antibody.

6. The method according to claim 5, wherein the anti-PD-1 monoclonal antibody is pembrolizumab.

7. The method according to claim 1, wherein the combination of the asparagine inhibitor and the immune checkpoint inhibitor enhances efficacy of cancer immunotherapy by activating killer T cells.

8. The method according to claim 7, wherein the killer T cells are CD8⁺ T cells.

9. The method according to claim 1, wherein the asparagine inhibitor has an asparagine deprivation effect.

10. The method according to claim 9, wherein the asparagine deprivation enhances glutamine metabolism to enhance CD8⁺ T cell activation.

11. The method according to claim 9, wherein the asparagine deprivation tailors differentiation and metabolic adaptation in a reactive oxygen species (ROS)-dependent manner.

12. The method according to claim 9, wherein the asparagine deprivation enhances CD8⁺ T cell effector functions.

13. The method according to claim 1, wherein the pharmaceutical composition is in a dosage form for oral administration.

14. The method according to claim 1, wherein the pharmaceutical composition is in a dosage form for parenteral administration.

15. A pharmaceutical composition for treating cancer, comprising a combination of an asparagine inhibitor and an immune checkpoint inhibitor (ICI).

16. The pharmaceutical composition according to claim 15, wherein the asparagine inhibitor is L-asparaginase.

17. The pharmaceutical composition according to claim 15, wherein the immune checkpoint inhibitor is an anti-PD-1 monoclonal antibody.

18. The pharmaceutical composition according to claim 17, wherein the anti-PD-1 monoclonal antibody is pembrolizumab.
